(19) United States
(12) Patent Application Publication
(10) Pub. No.: US 2013/0085157 A1
(43) Pub. Date: Apr. 4, 2013

(54) ARYLSUBSTITUTED THIAZOLOTRIAZOLES AND THIAZOLOIMIDAZOLES

(71) Applicants: Emilie D. Smith, Apex, NC (US); Mark E. Fitzgerald, Durham, NC (US); Rong Jiang, Fuquay Varina, NC (US); Robert N. Atkinson, Raleigh, NC (US); James M. Veal, Apex, NC (US); Kenneth H. Huang, Chapel Hill, NC (US)

(72) Inventors: Emilie D. Smith, Apex, NC (US); Mark E. Fitzgerald, Durham, NC (US); Rong Jiang, Fuquay Varina, NC (US); Robert N. Atkinson, Raleigh, NC (US); James M. Veal, Apex, NC (US); Kenneth H. Huang, Chapel Hill, NC (US)

(21) Appl. No.: 13/630,946

(22) Filed: Sep. 28, 2012

Related U.S. Application Data
(60) Provisional application No. 61/541,741, filed on Sep. 30, 2011.

(51) Int. Cl. A61K 31/429; A61K 31/4725; A61P 9/10; C07D 513/04

(52) U.S. Cl. 514/307; 548/154; 514/368; 546/148

(57) ABSTRACT

This disclosure relates to compounds, compositions and methods for the treatment of various disorders. In particular, the disclosure relates to thiazolotriazole and thiazoloimidazole compounds which agonize the activity of the protein TGR5.

### CROSS-REFERENCE TO RELATED APPLICATIONS

This application claims benefit of U.S. Provisional Application Ser. No. 61/541,741, filed Sep. 30, 2011, the disclosure of which is incorporated by reference herein in its entirety.

### BACKGROUND

1. Field of the Disclosure

This disclosure relates to compounds, compositions and methods for the treatment of various disorders. In particular, the disclosure relates to compounds which agonize the activity of the protein TGR5.

2. Description of Related Art

Diabetes mellitus is now a major health epidemic. The CDC currently estimates that diabetes affects approximately 26 million individuals in the United States, representing 8.3% of the population. Additionally, some 79 million individuals are estimated to be pre-diabetic based on glucose and hemoglobin A1c levels. Diabetes is the 7th leading cause of death and the leading cause of kidney failure, non-traumatic lower limb amputations, and new cases of blindness in adults. It is a major contributor to heart disease and stroke, strongly correlated with obesity, and a central component of the metabolic syndrome.

The large majority (>90%) of diabetes cases are type II diabetes in which high blood glucose levels are a result of insulin resistance (insensitivity to insulin) as well as possible insulin deficiency. Type II diabetes may be treated in multiple ways including diet, exercise, insulin, and pharmaceutical therapy. Greater than 70% of diabetics utilize some sort of pharmaceutical therapy. Example therapies currently include metformin (glucose lowering agent), sulphonyl ureas (enhanced insulin production in pancreas), agonists of peroxisome proliferator activated receptors (enhanced insulin action), and alpha-glucosidase inhibitors (lowered glucose production). These agents are often effective but can have a number of side effects including hypoglycemia, gastrointestinal issues, weight gain and edema. Additionally, effectiveness of the agent may diminish over time. More recently, medicines that cause elevation of Glucagon-like peptide-1 (GLP-1) have been pursued as anti-diabetic agents. An increase in circulating GLP-1 levels has been shown to have an incretin effect, raising insulin levels. Medicines that are peptide analogs of GLP-1 or increase GLP-1, e.g. DPP-IV inhibitors, have proved beneficial.

Despite these available therapies, the incidence of diabetes has continued to increase as described above. As noted, current therapies may be of limited effectiveness, lose effectiveness over time, or have undesirable side effects that limit their use. Thus, there still remains a clear need for new diabetic medications, particular those that function through novel cellular mechanisms.

Bile acids have long been known to play a central role in the digestive process. Bile acids are amphipathic molecules that are synthesized from cholesterol in the liver, stored in the gall bladder, and secreted to the duodenum during the digestive process. In the duodenum, their function includes the critical role of solubilizing dietary fats and some vitamins, thereby facilitating and permitting absorption of these materials. Recently, cellular receptors have been identified that specifically bind bile acids. These receptors are the nuclear hormone receptor, farnesoid X receptor alpha (FXR) and the G-protein coupled receptor (GPCR) known as TGR5. TGR5 is also referred to in the literature as GPBAR1, M-BAR, GPR131, and BG37. Through signaling effects at these receptors, bile acids may in fact be able to regulate a number of cellular processes including those related to their own enterohepatic circulation, but also processes relevant to glucose, cholesterol, and triglyceride metabolism.

TGR5 is found to be widely expressed on the surface of enteroendocrine L-cells in the distal intestine. Evidence has shown clearly that these cells secrete GLP-1, as well as other putative incretins such as PYY. Moreover, it has been demonstrated that the secretion of GLP-1 and PYY in these cells is induced by agonism of TGR5 via a signaling pathway that utilizes cAMP. In short, compelling evidence shows that agonism of the TGR5 receptor leads to cAMP production which in turn leads to an increase in circulating GLP-1 and other possibly beneficial incretins. In vivo data from diabetic and obesity animal models demonstrate that beneficial therapeutic effects are observed following activation of TGR5.

Therefore, given that there is a distinct and great need for new diabetic medications, and that agonism of TGR5 receptor provides a novel strategy towards that end, identification of small molecules that are agonists of TGR5 is of high interest.

### SUMMARY OF THE DISCLOSURE

In a broad aspect, the disclosure encompasses compounds of formula (I), shown below, pharmaceutical compositions containing those compounds and methods of using such compounds to agonize the activity of the protein TGR5.

The compounds of the invention bind and activate TGR5. They are therefore useful for treating diseases and disorders where activation of the TGR5 receptor is beneficial; such diseases include diabetes, metabolic syndrome, obesity, dyslipidemia, inflammatory diseases (chronic and acute), and hypercholesterolemia.

Thus, one aspect (embodiment 1) of the disclosure provides compounds of formula (I):

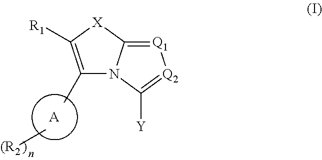

or a pharmaceutically acceptable salt thereof, wherein
X is O, S, or N(R₆);
Y is −OR₃, —O—Z—R₃, —SR₃, —S—Z—R₃, —N(R₆)R₃, —N(R₆)—Z—R₃, or —Z—R₃;
Z is —C(R₄)(R₅)—, or —C(R₄)(R₅)—C(R₄)(R₅)—;
Q₁ and Q₂ are independently N or CH, provided that at least one of Q₁ and Q₂ is N;
A is phenyl, naphthyl, pyridinyl, pyrazinyl, pyrimidinyl, or pyridazinyl;
n is 0, 1, 2, 3, or 4;
R₁ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, aryl, aryl C₁-C₆ alkyl, aryl C₁-C₆ alkenyl, heteroaryl, heteroaryl C₁-C₆ alkyl, heteroaryl C₁-C₆ alkenyl, —CON(C₁-C₆ alkyl)-aryl, —CONH-aryl, —O-aryl, —O—CH₂-aryl, —O—(CH₂)₂-aryl, —NH-aryl, —NH—CH₂-aryl, —NH—(CH₂)₂-aryl, —S-aryl, —S—CH₂-aryl, or —S—(CH₂)₂-aryl, wherein each is optionally substituted at any suitable position with one or more of R₇; or
each R₂ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —CONH₂, —CON(C₁-C₆ alkyl), —NHCO(C₁-C₆ alkyl), —N(C₁-C₆ alkyl)CO(C₁-C₆ alkyl), —CON(C₁-C₆ alkyl)₂, —CONH—OH, —CONH—NH₂, —CO₂H, —CO₂(C₁-C₆ alkyl), —OCO(C₁-C₆ alkyl), C₃-C₈ cycloalkyl, aryl, heteroaryl, and heterocyclyl;
R₃ is hydrogen, C₃-C₈ cycloalkyl, aryl, heteroaryl, or heterocyclyl, wherein each is optionally substituted with one or more of R₈;
each R₄ and R₅ independently are hydrogen, C₁-C₆ alkyl, or C₁-C₆ haloalkyl;
R₆ is hydrogen or C₁-C₆ alkyl;
R₇ is halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, or C₁-C₆ haloalkoxy; and
R₈ is halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —CONH₂, —CON(C₁-C₆ alkyl), —NHCO(C₁-C₆ alkyl), —N(C₁-C₆ alkyl)CO(C₁-C₆ alkyl), —CON(C₁-C₆ alkyl)₂, —CONH—OH, —CONH—NH₂, —CO₂H, —CO₂(C₁-C₆ alkyl), —OCO(C₁-C₆ alkyl), C₃-C₈ cycloalkyl, aryl, heteroaryl, or heterocyclyl;
   provided the compound is not:
5-phenyl-3-(phenylmethyl)-thiazolo[2,3-c]-1,2,4-triazole;
5-(4-chlorophenyl)-3-(phenylmethyl)-thiazolo[2,3-c]-1,2,4-triazole;
5-(4-bromophenyl)-3-(phenylmethyl)-thiazolo[2,3-c]-1,2,4-triazole;
3-anilino-5,6-diphenyl-thiazolo[2,3-c]-s-triazole;
3-(benzylthio)-5,6-diphenyl-thiazolo[2,3-c]-s-triazole;
5-(4-fluorophenyl)-3-[(3-pyridinylmethyl)thio]-thiazolo[2,3-c]-1,2,4-triazole;
3-[(1,3-benzodioxol-5-ylmethyl)thio]-5-phenyl-thiazolo[2,3-c]-1,2,4-triazole;
5-(4-methylphenyl)-3-[[(2-methyl-4-thiazolyl)methyl]thio]-thiazolo[2,3-c]-1,2,4-triazole;
5-(4-chlorophenyl)-3-[[(3,5-dimethyl-4-isoxazolyl)methyl]thio]-thiazolo[2,3-c]-1,2,4-triazole;
3-[[(3,5-dimethyl-4-isoxazolyl)methyl]thio]-5-phenyl-thiazolo[2,3-c]-1,2,4-triazole;
5-(4-chlorophenyl)-3-[[(2-methyl-4-thiazolyl)methyl]thio]-thiazolo[2,3-c]-1,2,4-triazole;
3-[[(2-methyl-4-thiazolyl)methyl]thio]-5-phenyl-thiazolo[2,3-c]-1,2,4-triazole; or
3-anilino-5,6-diphenyl-oxazolo[2,3-c]-s-triazole.

The disclosure also provides pharmaceutical compositions comprising a compound of formula (I) and at least one pharmaceutically acceptable carrier, solvent, adjuvant or diluent.

The disclosure also provides synthetic intermediates that are useful in making the compounds of formula (I).

The disclosure also provides methods of preparing compounds of the disclosure and the intermediates used in those methods.

The disclosure further provides a compound or pharmaceutical composition thereof in a kit with instructions for using the compound or composition.

In addition, the disclosure provides methods for treating obesity or type II diabetes, the method comprising administering to a subject in need of such treatment an effective amount of one or more compounds of formula (I).

The disclosure further provides methods for treating obesity or type II diabetes comprising co-administering a second anti-diabetic drug. In certain aspects, the second anti-diabetic drug is a sulfonylurea, meglitinide, biguanide, alpha-glucosidase inhibitor, glucagon-like peptide-1 analog or agonist, amylin analogue, dipeptidyl peptidase-4 inhibitor, thiazolidinediones or glitazone.

The disclosure also provides methods of lowering blood glucose, the method comprising administering to a subject in need of such treatment an effective amount of one or more compounds of formula (I).

The disclosure further provides methods for enhancing insulin secretion, the method comprising administering to a subject in need of such treatment an effective amount of one or more compounds of formula (I).

### DETAILED DESCRIPTION

In one embodiment, the disclosure provides compounds of formula (I) that may be represented by the formula:

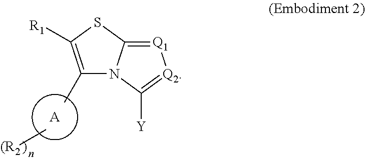

In embodiment 3, which is based on formula (I), the compounds may have the formula:

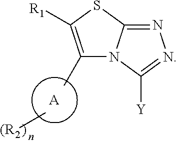

Embodiment 4, which is based on formula (I), provides compounds of that may be represented by the formula:

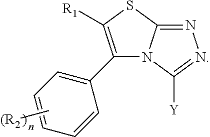

The compounds of formula (I) may also have the following formula in embodiment 5:

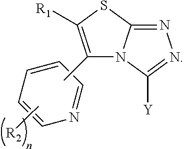

In another embodiment, the disclosure provides compounds of formula (I) that may be represented by the formulae:

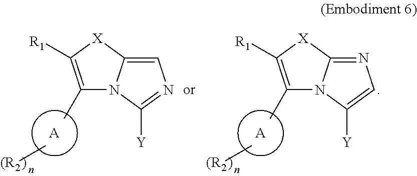

In another embodiment, the disclosure provides compounds of formula (I) that may be represented by the formulae:

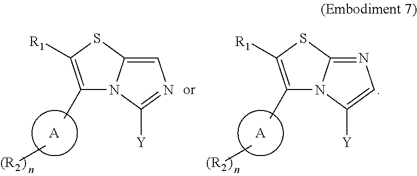

In embodiment 8, the disclosure provides compounds of formula (I) that may be represented by the formulae:

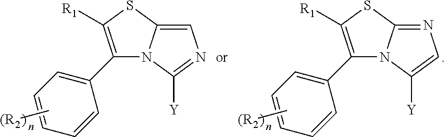

Embodiment 9 provides compounds of formula (I) that may be represented by the formulae:

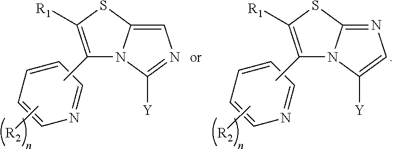

Another embodiment of the invention, i.e., embodiment 10, encompasses compounds of any of embodiments 1-9 where:
R₁ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, aryl, aryl C₁-C₆ alkyl, aryl C₁-C₆ alkenyl, heteroaryl, heteroaryl C₁-C₆ alkyl, —CON(C₁-C₆ alkyl)-aryl, —CONH-aryl, —O—OH₂-aryl, —NH—CH₂-aryl, or —S—CH₂-aryl, wherein each is optionally substituted at any suitable position with one or more of R₇.

Particular embodiments based on formula (I) include those of embodiment 11, i.e., compounds of embodiment 10 wherein R₁ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, aryl, aryl C₁-C₆ alkyl, aryl C₁-C₆ alkenyl, heteroaryl, or heteroaryl C₁-C₆ alkyl, wherein each is optionally substituted at any suitable position with one or more of R₇.

Some embodiments based on formula (I) include those of embodiment 12, wherein the compounds of embodiment 11 have R₁ that is hydrogen, halogen, C₁-C₆ alkyl, aryl, aryl C₁-C₆ alkyl, aryl C₁-C₆ alkenyl, heteroaryl, or heteroaryl C₁-C₆ alkyl, wherein each is optionally substituted at any suitable position with one or more of R₇.

In embodiment 13, the compounds based on formula (I) include those of embodiment 12 wherein R₁ is hydrogen, halogen, or C₁-C₆ alkyl. Embodiment 14 provides compounds of embodiment 13 where R₁ is hydrogen.

In embodiment 15, the compounds based on formula (I) include those of embodiment 12 wherein R₁ is aryl, aryl C₁-C₆ alkyl, or aryl C₁-C₆ alkenyl, wherein each is optionally substituted at any suitable position with one or more of R₇.

Some embodiments based on formula (I) include those of embodiment 16, wherein the compounds of embodiment 15 have R₁ that is aryl or aryl C₁-C₆ alkyl, wherein each is optionally substituted at any suitable position with one or more of R₇. Other embodiments based on formula (I) include those of embodiment 17, wherein the compounds of embodiment 16 have R₁ that is aryl optionally substituted at any suitable position with one or more of R₇, where R₇ is halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, or C₁-C₆ haloalkoxy. In embodiment 18, R₁ is aryl optionally substituted at any suitable position with one or more of R₇, where R₇ is halogen, C₁-C₆ alkyl, —OH, or C₁-C₆ alkoxy.

Other embodiments based on formula (I) include those of embodiment 19, wherein the compounds of embodiment 16 have R₁ that is aryl C₁-C₆ alkyl optionally substituted at any suitable position with one or more of R₇, where R₇ is halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, or C₁-C₆ haloalkoxy. In embodiment 20, R₁ is aryl C₁-C₆ alkyl optionally substituted at any suitable position with one or more of R₇, where R₇ is halogen, C₁-C₆ alkyl, —OH, or C₁-C₆ alkoxy.

Particular embodiments based on formula (I) include those of embodiment 21, i.e., compounds of embodiment 10 wherein R₁ is —CON(C₁-C₆ alkyl)-aryl, —CONH-aryl, —O—CH₂-aryl, —NH—CH₂-aryl, or —S—CH₂-aryl, wherein each is optionally substituted at any suitable position with one or more of R₇.

Particular embodiments based on formula (I) include those of embodiment 22, i.e., compounds of embodiment 21 wherein R₁ is —CON(C₁-C₆ alkyl)-aryl, or —S—CH₂-aryl, wherein each is optionally substituted at any suitable position with one or more of R₇.

Some embodiments based on formula (I) include those of embodiment 23, wherein the compounds of embodiment 21 or 23 have R₇ that is halogen, C₁-C₆ alkyl, —OH, or C₁-C₆ alkoxy.

Another embodiment of the invention, i.e., embodiment 24, encompasses compounds of any of embodiments 1-23 where n is 0, 1, 2, or 3; and
each R₂ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —CONH₂, —CON(C₁-C₆ alkyl), —NHCO(C₁-C₆ alkyl), —N(C₁-C₆ alkyl)CO(C₁-C₆ alkyl), —CON(C₁-C₆ alkyl)₂, —CO₂H, —CO₂(C₁-C₆ alkyl), —OCO(C₁-C₆ alkyl), C₃-C₈ cycloalkyl, aryl, heteroaryl, and heterocyclyl.

Some embodiments based on formula (I) include those of embodiment 25, wherein the compounds of embodiment 24 have each R₂ that is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —NHCO(C₁-C₆ alkyl), —N(C₁-C₆ alkyl)CO(C₁-C₆ alkyl), C₃-C₈ cycloalkyl, aryl, and heteroaryl.

Particular embodiments based on formula (I) include those of embodiment 26, i.e., compounds of embodiment 25 wherein each R₂ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —NHCO(C₁-C₆ alkyl), and —N(C₁-C₆ alkyl)CO(C₁-C₆ alkyl).

Particular embodiments based on formula (I) include those of embodiment 27, i.e., compounds of embodiment 25 wherein each R₂ is independently selected from the group consisting of C₃-C₈ cycloalkyl, aryl, and heteroaryl.

Particular embodiments based on formula (I) include those of embodiment 28, i.e., compounds of embodiment 25 wherein each R₂ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, and C₁-C₆ alkoxy. In embodiment 29, each R₂ is independently selected from the group consisting of halogen, C₁-C₆ alkyl, and C₁-C₆ alkoxy.

Particular embodiments based on formula (I) include those of embodiment 30, i.e., compounds of embodiment 28 wherein n is 0, 1, 2, or 3; and each R₂ is independently halogen or C₁-C₆ alkoxy.

Some embodiments based on formula (I) include those of embodiment 31, wherein the compounds of embodiment 24 have n that is 0.

Another embodiment of the invention, i.e., embodiment 32, encompasses compounds of any of embodiments 1-31 where Y is −OR₃, —O—Z—R₃, —SR₃, —S—Z—R₃, —N(R₆)R₃, or —N(R₆)—Z—R₃; and Z is —C(R₄)(R₅)—, or —C(R₄)(R₅)—C(R₄)(R₅)—.

Particular embodiments based on formula (I) include those of embodiment 33, i.e., compounds of embodiment 32 wherein Y is —SR₃ or —S—Z—R₃; and Z is —C(R₄)(R₅)—, or —C(R₄)(R₅)—C(R₄)(R₅)—.

Some embodiments based on formula (I) include those of embodiment 34, i.e., compounds of embodiment 33 wherein Y is —SR₃; and R₃ is C₃-C₈ cycloalkyl, aryl, heteroaryl, or heterocyclyl, wherein each is optionally substituted with one or more of R₈.

Another embodiments based on formula (I) include those of embodiment 35, i.e., compounds of embodiment 34 wherein Y is —SR₃; and R₃ is aryl or heteroaryl, wherein each is optionally substituted with one or more of R₈.

Particular embodiments based on formula (I) include those of embodiment 36, i.e., compounds of embodiment 33 wherein Y is —S—Z—R₃; and Z is —C(R₄)(R₅)—, or —C(R₄)(R₅)—C(R₄)(R₅)—, and R₃ is C₃-C₈ cycloalkyl, aryl, heteroaryl, or heterocyclyl, wherein each is optionally substituted with one or more of R₈.

Some embodiments based on formula (I) include those of embodiment 37, i.e., compounds of embodiment 26 wherein Y is —S—Z—R₃, Z is —C(R₄)(R₅)—, and R₃ is aryl, heteroaryl, or heterocyclyl, wherein each is optionally substituted with one or more of R₈.

Another embodiments based on formula (I) include those of embodiment 38, i.e., compounds of embodiment 37 wherein Y is —S(CH₂)₂—R₃, and R₃ is aryl, heteroaryl, or heterocyclyl, wherein each is optionally substituted with one or more of R₈.

Particular embodiments based on formula (I) include those of embodiment 39, i.e., compounds of embodiment 32 wherein Y is —OR₃ or —O—Z—R₃; and Z is —C(R₄)(R₅)—, or —C(R₄)(R₅)—C(R₄)(R₅)—.

Some embodiments of formula (I) include those of embodiment 40, wherein the compounds of embodiment 39 are those wherein Y is —OR₃; and R₃ is C₃-C₈ cycloalkyl, aryl, heteroaryl, or heterocyclyl, wherein each is optionally substituted with one or more of R₈. Other embodiments of formula (I) include those of embodiment 41, wherein Y is —OR₃; and R₃ is aryl or heteroaryl, wherein each is optionally substituted with one or more of R₈.

Some embodiments of formula (I) include those of embodiment 42, wherein the compounds of embodiment 39 are those wherein Y is —O—Z—R₃; and Z is —C(R₄)(R₅)—, or —C(R₄)(R₅)—C(R₄)(R₅)—, and R₃ is C₃-C₈ cycloalkyl, aryl, heteroaryl, or heterocyclyl, wherein each is optionally substituted with one or more of R₈.

Other embodiments of formula (I) include those of embodiment 43, wherein the compounds of embodiment 42 are those wherein Y is —O—Z—R₃, Z is —C(R₄)(R₅)—, and R₃ is aryl, heteroaryl, or heterocyclyl, wherein each is optionally substituted with one or more of R₈.

Additional embodiments of formula (I) include those of embodiment 44, wherein the compounds of embodiment 43 are those wherein Y is —O(CH₂)₂—R₃, and R₃ is aryl, heteroaryl, or heterocyclyl, wherein each is optionally substituted with one or more of R₈.

Particular embodiments based on formula (I) include those of embodiment 45, i.e., compounds of embodiment 32 wherein Y is —N(R₆)R₃, or —N(R₆)—Z—R₃; and Z is —C(R₄)(R₅)—, or —C(R₄)(R₅)—C(R₄)(R₅)—.

Some embodiments of formula (I) include those of embodiment 46, wherein the compounds of embodiment 45 are those wherein Y is —N(R₆)R₃; and R₃ is aryl or heteroaryl, wherein each is optionally substituted with one or more of R₈.

Other embodiments of formula (I) include those of embodiment 47, wherein the compounds of embodiment 46 are those wherein Y is —N(R₆)—Z—R₃, Z is —C(R₄)(R₅)—, and R₃ is aryl, heteroaryl, or heterocyclyl, wherein each is optionally substituted with one or more of R₈.

Another embodiment of the invention, i.e., embodiment 48, encompasses compounds of any of embodiments 32-47 where R₃ is aryl optionally substituted with one or more of R₈. In embodiment 49, R₃ is phenyl optionally substituted with one or more of R₈. In embodiment 50, R₃ is benzo[d][1,3]dioxolyl, 2,3-dihydrobenzo[b][1,4]dioxanyl, or 2,3-dihydroindenyl, optionally substituted with one or more of R₈.

Another embodiment of the invention, i.e., embodiment 51, encompasses compounds of any of embodiments 32-47 where R₃ is heteroaryl optionally substituted with one or more of R₈.

Particular embodiment of the invention, i.e., embodiment 52, encompasses compounds of any of embodiments 32-51, wherein R₈ is halogen, —NO₂, —CN, C₁-C₆ alkyl, C₁-C₆ haloalkyl, —NH₂, —NH(C₁-C₆ alkyl), —N(C₁-C₆ alkyl)₂, —OH, and C₁-C₆ alkoxy.

One embodiment of the invention, i.e., embodiment 53, encompasses compounds of any of embodiments 1-31 where Y is —Z—R₃; and Z is —C(R₄)(R₆)— or —C(R₄)(R₆)—C(R₄)(R₆)—.

Some embodiments based on formula (I) include those of embodiment 54, wherein the compounds of embodiment 53 have Z that is —CH₂—, or —(CH₂)₂—.

Additional embodiments based on formula (I) include those of embodiment 55, wherein the compounds of embodiment 53 or 54 have R₃ that is hydrogen.

In embodiment 56, the compounds based on formula (I) include those of embodiment 53 or 54 wherein R₃ is aryl optionally substituted with one or more of R₈.

In embodiment 57, the compounds based on formula (I) include those of embodiment 53 or 54 wherein R₃ is heteroaryl optionally substituted with one or more of R₈.

### Therapeutic Applications

The methods, compounds, and pharmaceutical compositions of the present invention relate to agonists of the TGR5 receptor. These TGR5 agonists can exert their effect independently of natural bile acid ligands for the receptor. The methods, compounds, and pharmaceutical compositions of the present invention may be used to regulate or activate the TGR5 signaling pathway, either in vitro or in vivo; examples of such application are the elevation of intracellular cAMP and the increase of circulating GLP-1.

One aspect of the disclosure relates to increasing TGR5 pathway signaling activity in a sample, either in vitro or in vivo, utilizing a compound or pharmaceutical composition described in this disclosure. The sample may be in one of many forms. Examples of the sample, as used herein, include, without limitation TGR5 signaling pathway components in a recombinant cellular system, in a purified sample, in a partially purified sample, in cultured cells, in cellular extracts, in biopsied cells and extracts thereof, in bodily fluids (e.g. blood, serum, urine, feces, saliva, semen, tears) and extracts thereof. For example, a method of the invention can involve contacting a cell, in vitro or in vivo, with a TGR5 receptor agonist.

In certain embodiments, compounds, and pharmaceutical compositions of the present invention are agonists that cause activation of TGR5 receptor signaling by binding to TGR5. In certain embodiments, proteins in the TGR5 signaling pathway that are downstream of TGR5 (e.g. cAMP, GLP-1, and PYY) are also modulated in a cell, either in vitro or in vivo, in addition to the activation of TGR5. For example, the synthesis, expression, regulatory state, stabilization, cellular location, and/or activity of GLP-1 and PYY may be modulated.

Another embodiment of the disclosure provides for treating a patient by administering to the patient a compound or pharmaceutical composition described in the disclosure. The treated patient may have a disorder, show symptoms of a disorder, or be at risk of developing a disorder or recurrence of a disorder. Treatment of the patient can cure, remedy, or heal the patient of the disorder. Alternatively, treatment of the patient can prevent, alleviate, diminish, palliate or improve the disorder. Alternatively, treatment of the patient can affect or alter the symptoms of the disorder or predisposition toward the disorder. The disorders that can be treated are those disorders in which activation of the TGR5 receptor signaling pathway inhibits progression of the disorder. For example, blood glucose levels can beneficially reduced as a result of TGR5 receptor agonism. Alternatively, TGR5 receptor agonism can lead to a reduction in inflammation associated with damage to pancreatic beta cells. Alternatively, TGR5 receptor agonism can improve insulin sensitivity in diseased cells or tissues. Alternatively, TGR5 receptor agonism can beneficially reduce lipid levels.

Relevant, non-limiting, disorders that can be treated by administering to the patient a compound or pharmaceutical composition described in the disclosure include: diabetes, including type 1 diabetes, type 2 diabetes, and gestational diabetes, impaired fasting glucose, impaired glucose tolerance, insulin resistance, hyperglycemia, obesity, metabolic syndrome, retinopathy, vascular restenosis, hypercholesterolemia, hypertriglyceridemia, dyslipidemia or hyperlipidemia, lipid disorders such as low HDL cholesterol or high LDL cholesterol, high blood pressure, angina pectoris, coronary artery disease, atherosclerosis, cardiac hypertrophy, ischemia, myocardial infarction, ulcerative colitis, crohn's disease, irritable bowel syndrome, fatty liver, non-alcoholic fatty liver disease, liver fibrosis, non-alcoholic steatohepatitis, liver cirrhosis, liver cholestasis, primary biliary cirrhosis, gall bladder stones, choledocholithiasis, cholecystitis, primary sclerosing cholangitis, rheumatoid arthritis, and kidney fibrosis.

The compounds and pharmaceutical compositions described in the disclosure are particularly useful for the treatment or prevention of metabolic disease. Relevant, non-limiting, metabolic diseases that can be treated or prevented by administering to the patient a compound or pharmaceutical composition described in the disclosure include: diabetes, diabetes, especially type 2 diabetes, and gestational diabetes, metabolic syndrome, impaired fasting glucose, impaired glucose tolerance, insulin resistance, obesity, hypercholesterolemia, and dyslipidemia. Treatment can be in either adults or children.

In some embodiments and for certain disorders, the TGR5 agonist described in the disclosure is used to treat the disorder in combination with another therapeutic agent already approved or recognized by appropriate governing authorities as suitable for treatment of the disorder. The TGR5 agonist of the disclosure may be administered in dosage form either separately or in a single combined dosage with the other therapeutic. When the TGR5 agonist of the disclosure and other agent are administered separately, they may be administered simultaneously or the TGR5 agonist may be administered first or the other therapeutic agent may be administered first. Non-limiting examples of these agents include anti-diabetic agents, modulators of glucose synthesis, modulators of glucose transport, modulators of glucose absorption and resorption, anti-obesity agents, anti-inflammatory agents, and anti-hypertensives. Non-limiting examples of these agents include insulin, insulin analogs, sulfonylureas, meglitinides, biguanides, alpha-glucosidase inhibitors, glucagon-like peptide-1 analogs and agonists, amylin analogues, dipeptidyl peptidase-4 inhibitors, peroxisome proliferator activated receptor agonists such as thiazolidinediones and glitazones, bile acid sequestrants, cholesterol biosynthesis inhibitors (e.g. HMG-CoA-reductase inhibitors), cholesterol absorption inhibitors, acyl-CoA:cholesterol acyltransferase (ACAT) inhibitors, sodium-dependent glucose cotransporters (SGLT1 and SGLT2) inhibitors, microsomal triglyceride transfer protein (MTP) inhibitors, ileal bile acid tranporter inhibitors, GPR40 agonists, GPR120 agonists, CETP inhibitors, glycogen phosphorylase inhibitors, protein tyrosine phosphatase-1B (PTP-1B) inhibitors, squalene epoxidase inhibitors, glucagon receptor modulators, glucokinase activators, glucose transporter-4 (GLUT4) modulators, diglyceride acyltransferase (DGAT1 and DGAT2) inhibitors glucosamine-fructose-6-phosphate aminotransferase (GFAT) inhibitors, fructose-1,6-bisphosphatase inhibitors, 11-beta-hydroxysteroid-dehydrogenase-1 inhibitors, acetyl-CoA carboxylase (ACC1 and ACC2) inhibitors, farnesoid X receptor (FXR) modulators, somatostatin 5 receptor (SST5) antagonists, glycogen synthase kinase-3 (GSK3) inhibitors, estrogen receptor agonists, and lipase inhibitors. Non-limiting examples of these agents include tolbutamide, acetohexamide, tolazamide, glipizide, glyburide, chlorpropamide, glibenclamide, glimepiride, gliclazide, gliquidone, repaglinide, nateglinidie, mitiglinide, metformin, phenformin, buformin, miglitol, acarbose, voglibose, epalrestat, exenatide, liraglutide, taspoglutide, albiglutide, pramlintide, vildagliptin, sitagliptin, saxagliptin, alogliptin, carmegliptin, denagliptin, rosiglitazone, pioglitazone, troglitazone, englitazone, balaglitazone, netoglitazone, lovastatin, simvastatin, pravastatin, fluvastatin, atorvastatin, cerivastatin, pitavastatin, nisvastatin, rivastatin, rosuvastatin, terbinafine, anacetrapib, torcetrapib, dalcetrapib, gemfibronzil, clofibrate, fenofibrate, bezafibrate, cholestyramine, colestipol, nicotinic acid, niacinamide, avasimibe, muraglitazar, naveglitazar, aleglitazar, tesaglitazar, peliglitazar, farglitazar, dapagliflozin, sergliflozin, midaglizole, isaglidole, deriglidole, idazoxan, efaroxan, fluparoxan, linogliride, nateglinide, repaglinide, mitiglinide, meglitinide, and salts thereof.

### Pharmaceutical Compositions

In another aspect, the present disclosure provides compositions comprising one or more of compounds as described above with respect to formula I and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

When used to treat or prevent such diseases, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as steroids, membrane stabilizers, 5LO inhibitors, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known.

For buccal administration, the compositions may take the form of tablets or lozenges formulated in conventional manner.

For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active metabolite compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective local dosages without undue experimentation.

### DEFINITIONS

The following terms and expressions used herein have the indicated meanings.

Terms used herein may be preceded and/or followed by a single dash, “—”, or a double dash, “═”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆alkoxycarbonyloxy and —OC(O)C₁-C₆alkyl indicate the same functionality; similarly arylalkyl and -alkylaryl indicate the same functionality.

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, —CH₂CH(CH₂CH₃)CH₂—.

The term “alkylene” refers to a bivalent alkyl group. An “alkylene chain” is a polymethylene group, i.e., —(CH₂)ₙ—, wherein n is a positive integer, preferably from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or this.

An “aralkyl” or “arylalkyl” group comprises an aryl group covalently attached to an alkyl group, either of which independently is optionally substituted. Preferably, the aralkyl group is aryl(C₁-C₆)alkyl, including, without limitation, benzyl, phenethyl, and naphthyl methyl.

The terms “cyano” and “nitrile” as used herein, mean a —CN group.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thia. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia.

The term “halo” or “halogen” as used herein, means —Cl, —Br, —I or —F.

The terms “haloalkyl”, “haloalkenyl” and “haloalkoxy” refer to an aliphatic, alkyl, alkenyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

The terms “heterocyclyl” and “heterocycloalkyl” as used herein, mean a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia.

The term “nitro” as used herein, means a —NO₂ group.

The term “oxo” as used herein means a ═O group.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

The term “thia” as used herein means a ═S group.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. Both the R and the S stereochemical isomers, as well as all mixtures thereof, are included within the scope of the disclosure.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

“Therapeutically effective amount” refers to that amount of a compound which, when administered to a subject, is sufficient to effect treatment for a disease or disorder described herein. The amount of a compound which constitutes a “therapeutically effective amount” will vary depending on the compound, the disorder and its severity, and the age of the subject to be treated, but can be determined routinely by one of ordinary skill in the art.

“Modulating” or “modulate” refers to the treating, prevention, suppression, enhancement or induction of a function, condition or disorder. For example, it is believed that the compounds of the present disclosure can modulate atherosclerosis by stimulating the removal of cholesterol from atherosclerotic lesions in a human.

“Treating” or “treatment” as used herein covers the treatment of a disease or disorder described herein, in a subject, preferably a human, and includes:

i. inhibiting a disease or disorder, i.e., arresting its development;

ii. relieving a disease or disorder, i.e., causing regression of the disorder;

iii. slowing progression of the disorder; and/or

iv. inhibiting, relieving, ameliorating, or slowing progression of one or more symptoms of the disease or disorder

“Subject” refers to a warm blooded animal such as a mammal, preferably a human, or a human child, which is afflicted with, or has the potential to be afflicted with one or more diseases and disorders described herein.

“EC₅₀” refers to a dosage, concentration or amount of a particular test compound that elicits a dose-dependent response at 50% of maximal expression of a particular response that is induced, provoked or potentiated by the particular test compound.

“IC₅₀” refers to an amount, concentration or dosage of a particular test compound that achieves a 50% inhibition of a maximal response in an assay that measures such response.

### Methods of Preparation

The compounds of the present disclosure may be prepared by use of known chemical reactions and procedures. Representative methods for synthesizing compounds of the disclosure are presented in the schemes bellow. It is understood that the nature of the substituents required for the desired target compound often determines the preferred method of synthesis. All variable groups of these methods are as described in the generic description if they are not specifically defined below.

Representative synthetic procedures for the preparation of compounds of the disclosure are outlined below in following schemes. Unless otherwise indicated, R₁, A, X, Y, Q₁, and Q₂ and carry the definitions set forth above in connection with the above formulae.

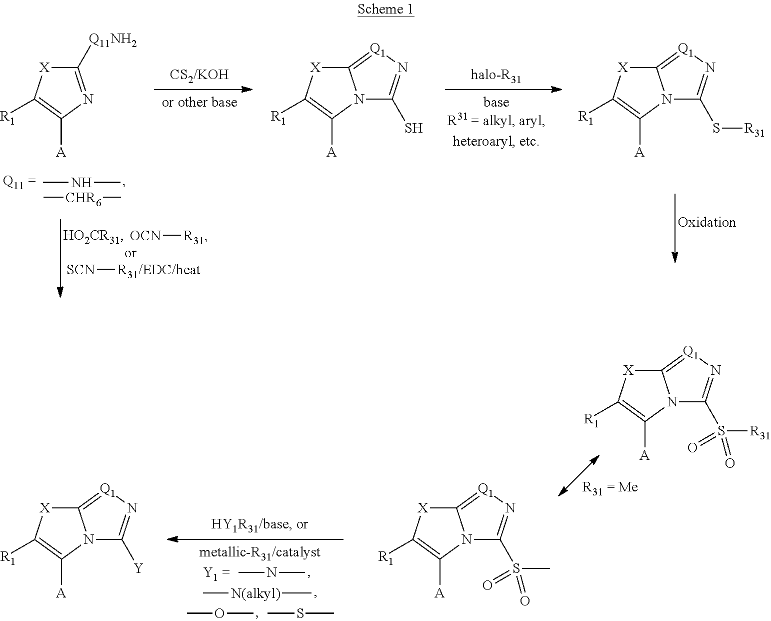

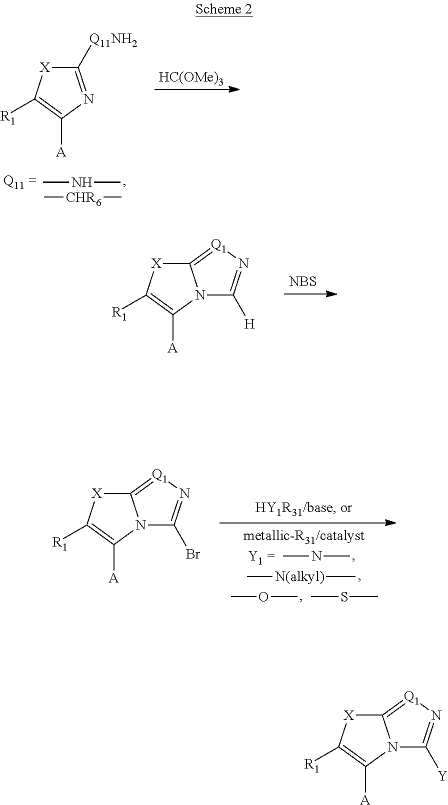

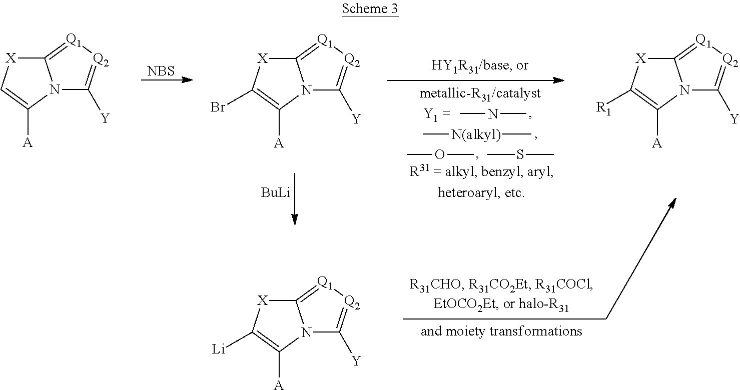

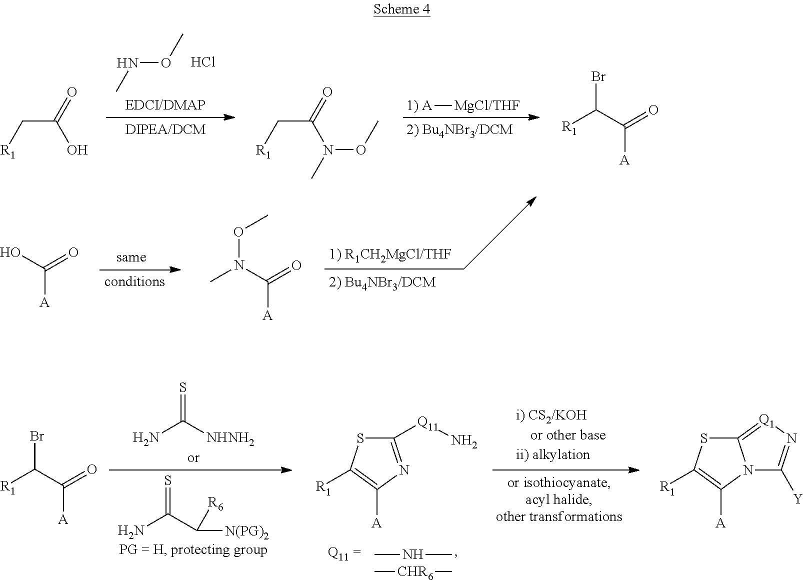

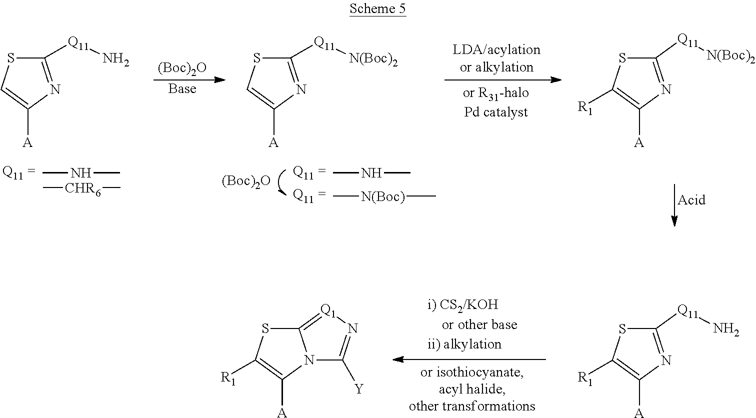

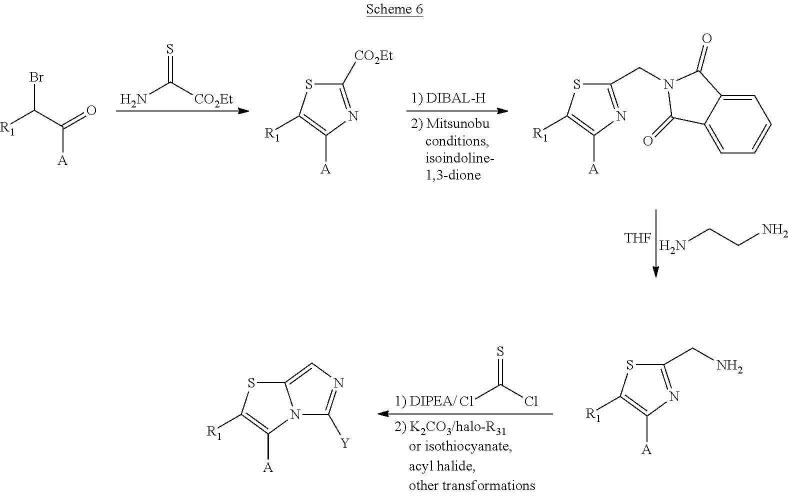

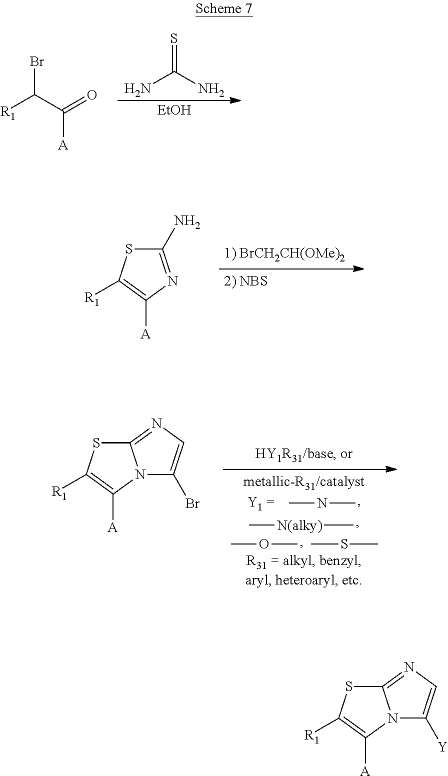

### General Procedure

Those having skill in the art will recognize that the starting materials and reaction conditions may be varied, the sequence of the reactions altered, and additional steps employed to produce compounds encompassed by the present disclosure, as demonstrated by the following examples. Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Starting materials can be obtained from commercial sources or prepared by well-established literature methods known to those of ordinary skill in the art. The reactions are performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure.

In some cases, protection of certain reactive functionalities may be necessary to achieve some of the above transformations. In general, the need for such protecting groups as well as the conditions necessary to attach and remove such groups will be apparent to those skilled in the art of organic synthesis. An authoritative account describing the many alternatives to the trained practitioner are J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4.sup.th edition, Vol. 15/I, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

Compound identity and purity confirmations are performed by LC/UV/MS using a Waters Micromass ZQ™ Detector and Waters 2695 Separations Module and Waters 2487 Dual I Absorbance Detector (Waters Corporation, Milford, Mass.). The diode array detector wavelength is 254 nm, and the MS is operated in positive electrospray ionization mode. The samples are maintained at room temperature in the autosampler, and an aliquot (5 μL) is injected onto an Ascentis Express C18 column, 30 mm×3 mm, 2.7 μm (Supelco Analytical, Bellefonte, Pa.) maintained at 40° C. The samples are eluted at a flow rate of 1 mL/min with a mobile phase system composed of solvent A (water containing 0.1% formic acid) and B (acetonitrile containing 0.1% formic acid) with an isocratic gradient 90% A for 0.3 min, then with a linear gradient 10% B to 90% B in 3.6 min, and then isocratic for 0.4 min with 90% B. The column is equilibrated back to the initial conditions for 0.4 min before the next run. In a few instances which are indicated in the examples, a long method is used utilizing 10-minute as total run time. Compound polarized mass and retention time (t_{R}), relative UV absorption area are used to assess purity and identity. Further, NMR spectra are utilized to characterize key intermediates and compounds. Optionally, compound R_{f} values on silica TLC plates are measured.

The disclosures of all articles and references mentioned in this application, including patents, are incorporated herein by reference in their entirety.

### EXAMPLES

The preparation of the compounds of the disclosure is illustrated further by the following examples, which are not to be construed as limiting the disclosure in scope or spirit to the specific procedures and compounds described in them. In all cases, unless otherwise specified, the column chromatography is performed using a silica gel solid phase.

### Example 1

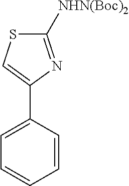

### t-butyl N-tert-butoxycarbonyl-N-[(4-phenylthiazol-2-yl)amino]carbamate

Commercially available (4-Phenylthiazol-2-yl)hydrazine (0.50 g, 2.62 mmol) in dichloromethane is added di-tert-butyl dicarbonate (0.57 g, 2.62 mmol) at 0° C. and the resulting mixture is stirred at room temperature for overnight. The solvent is removed in vacuo and the tert-butyl N-[(4-phenylthiazol-2-yl)amino]carbamate (0.49 g, 69%) is isolated by silica chromatograph. A mixture of this product (0.20 g, 0.68 mmol), Et₃N (0.18 mL, 1.22 mmol) and DMAP (10 mg, 0.08 mmol) in THF (2 mL) is added di-tert-butyl dicarbonate (0.22 g, 1.02 mmol) at 0° C. The resulting mixture is stirred at room temperature for 1 hour and concentrated in vacuo. The desired title compound is isolated by silica chromatograph (207 mg, 78%). LC/MS (m/z) M+Na=414.0; t_{R}=3.79 min.

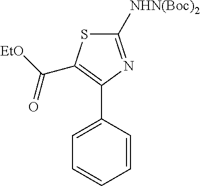

### Ethyl 2-[2,2-bis(tert-butoxycarbonyl)hydrazino]-4-phenyl-thiazole-5-carboxylate

n-BuLi (2.5 M in hexane, 1.3 mL, 3.22 mmol) is added to a solution of i-Pr₂NH in THF (10 mL) at 0° C. under nitrogen. One hour later, the reaction mixture is cooled down to −78° C. and a solution of t-butyl N-tert-butoxycarbonyl-N-[(4-phenylthiazol-2-yl)amino]carbamate (1.05 g, 2.68 mmol) in THF (3 mL) is added dropwise under nitrogen. The resulting mixture is stirred at −78° C. for 1 hour and is added a solution of ethyl chloroformate (0.36 mL, 3.75 mmol) in THF (3 mL) at −78° C. One hour later, the reaction mixture is warmed back to room temperature and quenched with saturated aqueous NH₄Cl solution followed by extraction with EtOAc (2×). The organic portions are separated, combined, dried with MgSO₄, filtered and concentrated in vacuo to give the desired title product that is used for next step without further purification. LC/MS (m/z) M+Na=486.4; t_{R}=4.02 min.

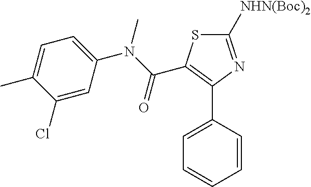

### di-tert-butyl 2-(5-((3-chloro-4-methylphenyl)(methyl)carbamoyl)-4-phenylthiazol-2-yl)hydrazine-1,1-dicarboxylate

A mixture of ethyl 2-[2,2-bis(tert-butoxycarbonyl)hydrazino]-4-phenyl-thiazole-5-carboxylate (1.30 g, 2.81 mmol) in a mixed solution of LiOH (1 N, 50 mL) and THF (50 mL) is stirred at room temperature for 2 days. The reaction mixture is neutralized with 1 N HCl to pH ˜7 and extracted with EtOAc (3×). The organic layers are separated, combined, dried with MgSO₄, filtered and concentrated in vacuo to give 2-[2,2-bis(tert-butoxycarbonyl)hydrazino]-4-phenyl-thiazole-5-carboxylic acid. LC/MS (m/z) M+Na=457.0; t_{R}=3.40 min. 2-Chloro-1-methylpyridinium iodide (0.42 g, 1.64 mmol) is added to a mixture of the carboxylic acid (0.60 g, 1.38 mmol), 3-chloro-N,4-dimethylaniline (0.26 g, 1.68 mmol) and Et₃N (0.48 mL, 3.45 mmol) in dichloromethane (3 mL) at room temperature in one portion. The resulting mixture is stirred at room temperature for 3 hours and concentrated in vacuo. The desired title compound (0.16 g, 22% yield) is isolated by silica chromatograph. LC/MS (m/z) M+Na=595.4; t_{R}=3.69 min.

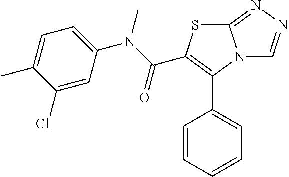 [SMILES: Cc1ccc(N(C)C(=O)c2sc3nncn3c2-c2ccccc2)cc1Cl]

### N-(3-chloro-4-methyl-phenyl)-N-methyl-5-phenyl-thiazolo[2,3-c][1,2,4]triazole-6-carboxamide

A mixture of tert-butyl N-tert-butoxycarbonyl-N-[[5-[(3-chloro-4-methyl-phenyl)-methyl-carbamoyl]-4-phenyl-thiazol-2-yl]amino]carbamate (0.16 g, 0.30 mmol) in HCl (4N in dioxane, 2 mL) is stirred at room temperature for overnight. The reaction mixture is basified with 1 N NaOH to pH ˜10 and extracted with EtOAc (2×). The organic layers are separated, combined, dried with MgSO₄, filtered and concentrated in vacuo to give N-(3-chloro-4-methyl-phenyl)-2-hydrazino-N-methyl-4-phenyl-thiazole-5-carboxamide (89 mg, 80%) which is used for next step without further purification. LC/MS (m/z) M+H=373.4; t_{R}=2.82 min.

A mixture of N-(3-chloro-4-methyl-phenyl)-2-hydrazino-N-methyl-4-phenyl-thiazole-5-carboxamide (0.04 g, 0.11 mmol) and trimethylorthoformate (2 mL) is stirred at 90° C. for 2 hours and concentrated in vacuo. To this residue is added HOAc (1 mL) and the resulting mixture is stirred at 90° C. for 2 hour. The HOAc is removed in vacuo and the desired title compound (0.02 g, 52%) is isolated by preparative HPLC. LC/MS (m/z) M+H=383.4; t_{R}=2.52 min.

### Example 2

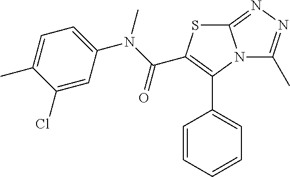

### N-(3-chloro-4-methyl-phenyl)-N,3-dimethyl-5-phenyl-thiazolo[2,3-c][1,2,4]triazole-6-carboxamide

This compound (0.01 g) is prepared by following the similar procedure as described above from N-(3-chloro-4-methyl-phenyl)-2-hydrazino-N-methyl-4-phenyl-thiazole-5-carboxamide and trimethylorthoacetate. LC/MS (m/z) M+H=397.4; t_{R}=2.65 min.

### Example 3

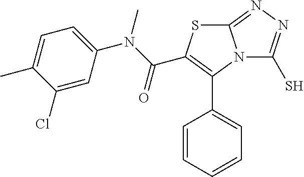 [SMILES: Cc1ccc(N(C)C(=O)c2sc3nnc(S)n3c2-c2ccccc2)cc1Cl]

### N-(3-chloro-4-methyl-phenyl)-N-methyl-5-phenyl-3-sulfanyl-thiazolo[2,3-c][1,2,4]triazole-6-carboxamide

To a mixture of N-(3-chloro-4-methyl-phenyl)-2-hydrazino-N-methyl-4-phenyl-thiazole-5-carboxamide and KOH (0.01 g, 0.16 mmol) in MeOH (5 mL) at room temperature is added CS₂ (0.09 mL, 1.5 mmol). The resulting mixture is refluxed at 80° C. for 8 hours and concentrated in vacuo. The residue is acidified with 1 N HCl to pH ˜2 and extracted with ether (3×). The organic layers are separated, combined, dried with MgSO₄, filtered and concentrated in vacuo to give desired title compound which is used for next step without further purification.

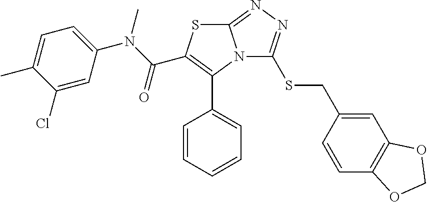 [SMILES: Cc1ccc(N(C)C(=O)c2sc3nnc(SCc4ccc5c(c4)OCO5)n3c2-c2ccccc2)cc1Cl]

### 3-(1,3-Benzodioxol-5-ylmethylsulfanyl)-N-(3-chloro-4-methyl-phenyl)-N-methyl-5-phenyl-thiazolo[2,3-c][1,2,4]triazole-6-carboxamide

To a mixture of N-(3-chloro-4-methyl-phenyl)-N-methyl-5-phenyl-3-sulfanyl-thiazolo[2,3-c][1,2,4]triazole-6-carboxamide (0.06 g, 0.14 mmol) and Et₃N (0.08 mL, 0.56 mmol) in dichloromethane (1 mL) at 0° C. is added 1,3-benzodioxol-5-ylmethyl methanesulfonate (0.07 mg, 0.28 mmol). The resulting mixture is stirred at room temperature for 1 hr and concentrated in vacuo. The desired title compound is isolated by preparative silica TLC as a solid (3.8 mg, 5%). LC/MS (m/z) M+H=519.4; M+Na=571.4; t_{R}=3.09 min.

### Example 4

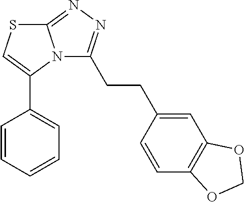 [SMILES: c1ccc(-c2csc3nnc(CCc4ccc5c(c4)OCO5)n23)cc1]

### 3-[2-(1,3-benzodioxol-5-yl)ethyl]-5-phenyl-thiazolo[2,3-c][1,2,4]triazole

A mixture of 3-(1,3-benzodioxol-5-yl)propanoic acid (0.10 g, 0.52 mmol), (4-phenylthiazol-2-yl)hydrazine (0.10 g, 0.52 mmol), EDC (0.15 g, 0.77 mmol), HOBt (0.07 g, 0.52 mmol) and K₂CO₃ (0.36 g, 2.58 mmol) in dichloromethane (2.5 mL) is stirred at room temperature for overnight. The reaction mixture is added water and extracted with dichloromethane (3×). The organic layers are separated, combined, dried with MgSO₄, filtered and concentrated in vacuo to give desired 3-(1,3-benzodioxol-5-yl)-W-(4-phenylthiazol-2-yl)propanehydrazide (0.22 g) which is used for next step without further purification. A mixture of this compound in POCl₃ (6 mL) is stirred at 100° C. for 2 hr and concentrated in vacuo. The resulting residue is neutralized with saturated aqueous NaHCO₃ to pH ˜7 and extracted with EtOAc. The organic layer is separated, dried with MgSO₄, filtered and concentrated in vacuo to give a crude product which is purified by silica chromatograph to afford the desired title compound (0.03 g, 18%). LC/MS (m/z) M+H=350.4; t_{R}=2.59 min.

### Example 5

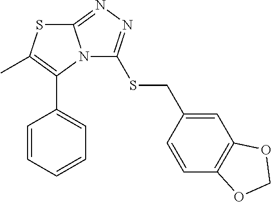 [SMILES: Cc1sc2nnc(SCc3ccc4c(c3)OCO4)n2c1-c1ccccc1]

### 3-(1,3-Benzodioxol-5-ylmethylsulfanyl)-6-methyl-5-phenyl-thiazolo[2,3-c][1, 2, 4]triazole

Hydrazinecarbothioamide (0.14 g, 1.5 mmol) is added to a solution of 2-bromo-1-phenyl-propan-1-one (0.32 g, 1.5 mmol) in EtOH (5 mL). The resulting mixture is refluxed for 1 hr and is added KOH (0.12 g, 1.5 mmol) followed by CS₂ (1.0 mL) at room temperature. The resulting mixture is refluxed for another 4 hr and concentrated in vacuo. 1 N NaOH (5 mL) is added to the residue and the solid is filtered off. The filtrate is acidified with 1 N HCl to pH ˜1. The desired product 6-methyl-5-phenyl-thiazolo[2,3-c][1,2,4]triazole-3-thiol (0.03 g, 10% yield) is collected by filtration and dried by lyophilization. LC/MS (m/z) M+H=248.2; t_{R}=2.36 min.

This intermediate is treated with 1,3-benzodioxol-5-ylmethyl methanesulfonate, Et₃N in dichloromethane at 0° C. for 1 hour as in the similar alkylation procedure described above to afford the desired title compound 3-(1,3-benzodioxol-5-ylmethylsulfanyl)-6-methyl-5-phenyl-thiazolo[2,3-c][1,2,4]triazole (0.03 g, 67% yield). LC/MS (m/z) M+H=382.3; t_{R}=2.84 min.

### Example 6

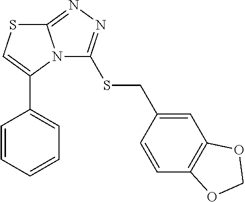 [SMILES: c1ccc(-c2csc3nnc(SCc4ccc5c(c4)OCO5)n23)cc1]

### 3-(1,3-Benzodioxol-5-ylmethylsulfanyl)-5-phenyl-thiazolo[2,3-c][1,2,4]triazole

To a mixture of (4-phenylthiazol-2-yl)hydrazine (4.0 g, 20.9 mmol) and KOH (1.2 g) in MeOH (100 mL) is added CS₂ (12 mL) at room temperature. The resulting mixture is refluxed at 70° C. for 4 hr and concentrated in vacuo. To the residue is added 1 N NaOH (80 mL), and the solid is filtered off. The filtrate is acidified with 1 N HCl to pH ˜1. The desired product 5-phenylthiazolo[2,3-c][1,2,4]triazole-3-thiol (4.8 g, 85%) is collected by filtration and dried by lyophilization. LC/MS (m/z) M+H=234.2; t_{R}=2.21 min.

This intermediate is treated with 1,3-benzodioxol-5-ylmethyl methanesulfonate, Et₃N in dichloromethane at 0° C. for 1 hour as using the similar alkylation procedure described above to afford the desired title compound 3-(1,3-benzodioxol-5-ylmethylsulfanyl)-5-phenyl-thiazolo[2,3-c][1,2,4]triazole (0.54 g, 87% yield). LC/MS (m/z) M+H=368.3; t_{R}=2.71 min.

### Example 7

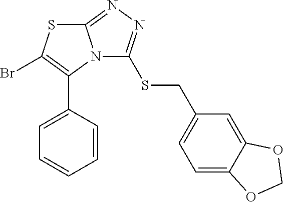 [SMILES: Brc1sc2nnc(SCc3ccc4c(c3)OCO4)n2c1-c1ccccc1]

### 3-(1,3-Benzodioxol-5-ylmethylsulfanyl)-6-bromo-5-phenyl-thiazolo[2,3-c][1,2,4]triazole

Bromine (0.11 mL, 2.2 mmol) is added dropwise to a solution of 3-(1,3-benzodioxol-5-ylmethylsulfanyl)-5-phenyl-thiazolo[2,3-c][1,2,4]triazole (0.54 g, 1.47 mmol) in CH₂Cl₂ (9 mL) at rt. The reaction mixture is stirred overnight and basified with saturated NaHCO₃ and extracted with EtOAc (3×). The organic portions are separated, combined and dried with MgSO₄, filtered and concentrated in vacuo to give a crude product which is purified by silics chromatograph to generate desired title compound (0.31 g, 47%). LC/MS (m/z) M+H=446.2, 448.2; t_{R}=2.94.

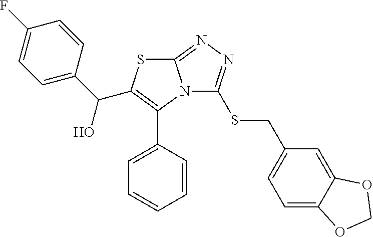 [SMILES: OC(c1ccc(F)cc1)c1sc2nnc(SCc3ccc4c(c3)OCO4)n2c1-c1ccccc1]

### [3-(1,3-Benzodioxol-5-ylmethylsulfanyl)-5-phenyl-thiazolo[2,3-c][1,2,4]triazol-6-yl]-(4-fluorophenyl)methanol

n-BuLi (2.5 M in hexane, 1.3 mL, 3.22 mmol) is added dropwise to a stirred solution of 3-(1,3-benzodioxol-5-ylmethylsulfanyl)-6-bromo-5-phenyl-thiazolo[2,3-c][1,2,4]triazole (67 mg, 0.15 mmol) in THF (2 mL) at −78° C. under nitrogen. The resulting mixture is stirred at −78° C. for 1 hr and a solution of 4-fluorobenzaldehyde (21 mg, 0.165 mmol) in THF (0.5 mL) is added dropwise. The resulting mixture is stirred at −78° C. for 2 hr, warmed back to room temperature slowly and quenched with saturated NH₄Cl followed by extraction with EtOAc (3×). The organic portions are separated, combined, dried with MgSO₄, filtered, concentrated in vacuo and the desired title compound (10 mg, 16%) is isolated by preparative HPLC. LC/MS (m/z) M+H=492.3; t_{R}=2.90 min.

### Example 8

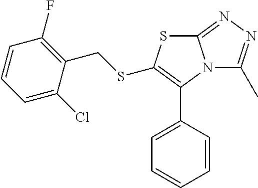 [SMILES: Cc1nnc2sc(SCc3c(F)cccc3Cl)c(-c3ccccc3)n12]

### 6-[(2-Chloro-6-fluoro-phenyl)methylsulfanyl]-3-methyl-5-phenyl-thiazolo[2,3-c][1,2,4]triazole

A mixture of 2-hydrazinyl-4-phenylthiazole (0.96 g, 5 mmol) and trimethylorthoformate (10 mL) is stirred at 95° C. for 2 hr and concentrated in vacuo. To this residue is added HOAc (1 mL) and the resulting mixture is stirred at 90° C. for 2 hr. The HOAc is remove in vacuo and the desired 3-methyl-5-phenyl-thiazolo[2,3-c][1,2,4]triazole (0.75 g, 70%) is isolated by silica chromatography. LC/MS (m/z) M+H=216.2; t_{R}=2.07 min.

Bromination of this intermediate with bromine in CH₂Cl₂ at rt for overnight the same way as described above affords 6-bromo-3-methyl-5-phenyl-thiazolo[2,3-c][1,2,4]triazole (120 mg, 41%). LC/MS (m/z) M+H=294.2, 296.2; t_{R}=2.32 min.

To a stirred mixture of 6-bromo-3-methyl-5-phenyl-thiazolo[2,3-c][1,2,4]triazole (30 mg, 0.1 mmol) and Cs₂CO₃ (65 mg, 0.2 mmol) in DMF (3 mL) at rt is added (2-chloro-6-fluoro-phenyl)methanethiol (36 mg, 0.2 mmol). The resulting mixture is stirred at 90° C. overnight and concentrated in vacuo. The desired 6-[(2-chloro-6-fluoro-phenyl)methyl-sulfanyl]-3-methyl-5-phenyl-thiazolo[2,3-c][1,2,4]triazole (2 mg, 5%) is isolated by preparative HPLC. LC/MS (m/z) M+H=390.3; t_{R}=2.96 min.

### Example 9

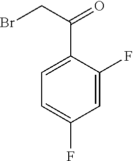 [SMILES: O=C(CBr)c1ccc(F)cc1F]

### 2-Bromo-1-(2,4-difluorophenyl)ethanone

Bromine (0.95 mL, 18.5 mmol) in HOAc (1 mL) is added dropwise to a solution of 1-(2,4-difluorophenyl)ethanone (2.88 g, 18.5 mmol) in HOAc (9 mL) at 0° C. After stirring at 0° C. for 20 min, the mixture is warmed back to room temperature and stirred for 1 hr. The reaction mixture is neutralized with saturated NaHCO₃ and extracted with EtOAc (3×). The organic portions are separated, combined and dried with MgSO₄, filtered and concentrated in vacuo to give the crude title product (4 g) that is used without further purification.

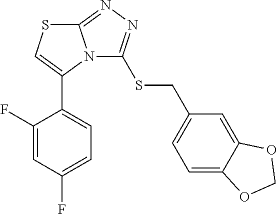 [SMILES: Fc1ccc(-c2csc3nnc(SCc4ccc5c(c4)OCO5)n23)c(F)c1]

### 3-(1,3-Benzodioxol-5-ylmethylsulfanyl)-5-(2,4-difluorophenyl)thiazolo[2,3-c][1,2,4]triazole

Hydrazinecarbothioamide (0.08 g, 0.85 mmol) is added to a solution of 2-bromo-1-(2,4-difluorophenyl)ethanone (0.2 g, 0.85 mmol) in EtOH (5 mL). The resulting mixture is refluxed for 1 hr and added KOH (0.06 g, 0.87 mmol) followed by CS₂ (0.5 mL) at room temperature. The resulting mixture is refluxed for another 4 hr and concentrated in vacuo. 1 N NaOH (5 mL) is added to the residue and the solid filtered off. The filtrate is acidified with 1 N HCl to pH ˜1. The desired product 5-(2,4-difluorophenyl)thiazolo[2,3-c][1,2,4]triazole-3-thiol (0.18 g, 80%) is collected by filtration and dried. LC/MS (m/z) M+H=270.2; t_{R}=2.24 min.

This intermediate is treated with 1,3-benzodioxol-5-ylmethyl methanesulfonate, Et₃N in dichloromethane at 0° C. for 1 hour using the similar alkylation procedure described above to afford the desired title compound 3-(1,3-Benzodioxol-5-ylmethylsulfanyl)-5-(2,4-difluorophenyl)thiazolo[2,3-c][1,2,4]triazole (0.02 g). LC/MS (m/z) M+H=404.3; t_{R}=2.72 min.

### Example 10

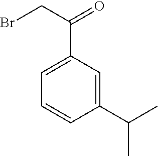

### 2-Bromo-1-(3-isopropylphenyl)ethanone

A mixture of N,O-dimethylhydroxylamine hydrochloride (0.44 g, 4.5 mmol), 3-isopropylbenzoic acid (0.5 g, 3 mmol), DMAP (0.18 g, 1.5 mmol), EDCI (0.76 g, 4 mmol), diisopropylethylamine (1.94 g, 15 mmol) in dichloromethane (5 mL) is stirred at room temperature for overnight. The reaction mixture is added water (10 mL) and extracted with dichloromethane (2×). The organic layers are separated, combined and washed with 1 N HCl followed by saturated NaHCO₃. The organic portion is dried with MgSO₄, filtered and concentrated in vacuo to give 3-isopropyl-N-methoxy-N-methyl-benzamide (0.5 g, 80%), which is used for next step without further purification.

A solution of CH₃MgBr (1.0 M in THF, 0.26 mL, 2.6 mmol) is added dropwise to a solution of 3-isopropyl-N-methoxy-N-methyl-benzamide (0.5 g, 2.4 mmol) in THF (5 mL) at 0° C. The reaction mixture is stirred at room temperature for 2 hr and quenched with saturated NH₄Cl followed by extraction with EtOAc (2×). The organic portions are separated, combined, dried with MgSO₄, filtered and concentrated in vacuo to give the desired product 1-(3-isopropylphenyl)ethanone (0.5 g) which is used directly without further purification.

This methyl ketone (0.5 g) is brominated with bromine in HOAc as described above to afford the title compound 2-bromo-1-(3-isopropylphenyl)ethanone (0.4 g) which is used for next step without further purification.

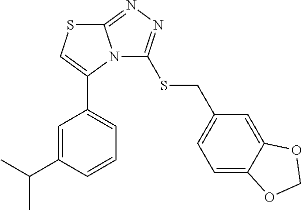 [SMILES: CC(C)c1cccc(-c2csc3nnc(SCc4ccc5c(c4)OCO5)n23)c1]

### 3-(1,3-Benzodioxol-5-ylmethylsulfanyl)-5-(3-isopropylphenyl)thiazolo[2,3-c][1,2,4]triazole

5-(3-isopropylphenyl)thiazolo[2,3-c][1,2,4]triazole-3-thiol (0.4 g) is prepared from 2-bromo-1-(3-isopropylphenyl)ethanone and hydrazinecarbothioamide using the same procedure described above. LC/MS (m/z) M+H=276.3; t_{R}=2.64 min.

This intermediate is treated with 1,3-benzodioxol-5-ylmethyl methanesulfonate, Et₃N in dichloromethane at 0° C. for 1 hour using the similar alkylation procedure described above to afford the desired title compound (0.03 g) as a solid. LC/MS (m/z) M+H=410.3, M+Na=432.3; t_{R}=3.09 min.

### Example 11

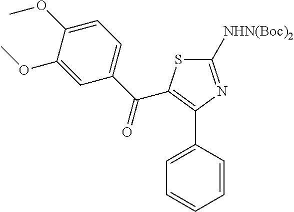

### t-Butyl-N-t-butoxycarbonyl-N-[[5-(3,4-dimethoxybenzoyl)-4-phenyl-thiazol-2-yl]amino]carbamate

The title compound is prepared from t-butyl-N-t-butoxycarbonyl-N-[(4-phenylthiazol-2-yl)amino]carbamate and 3,4-dimethoxybenzoyl chloride using LDA according to the same procedure described for Ethyl 2-[2,2-bis(tert-butoxycarbonyl)hydrazino]-4-phenyl-thiazole-5-carboxylate. LC/MS (m/z) M+Na=578.2; t_{R}=3.81 min.

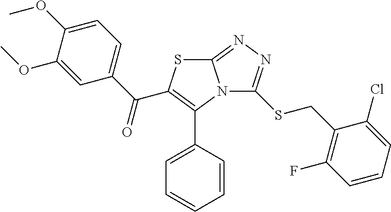 [SMILES: COc1ccc(C(=O)c2sc3nnc(SCc4c(F)cccc4Cl)n3c2-c2ccccc2)cc1OC]

### [3-[(2-Chloro-6-fluoro-phenyl)methylsulfanyl]-5-phenyl-thiazolo[2,3-c][1,2,4]triazol-6-yl]-(3,4-dimethoxyphenyl)methanone

(3,4-Dimethoxyphenyl)-(2-hydrazino-4-phenyl-thiazol-5-yl)methanone (1.4 g, 4 mmol) is prepared from t-butyl-N-t-butoxycarbonyl-N-[[5-(3,4-dimethoxybenzoyl)-4-phenyl-thiazol-2-yl]amino]carbamate via deprotection of the Boc groups using the same procedure described above. CS₂ (3 mL) is added to a mixture of this compound (1.4 g) and pyridine (3 mL) at room temperature. The resulting mixture is stirred for overnight and concentrated in vacuo. The residue is acidified with 1N HCl to pH ˜2 and extracted with dichloromethane (3×). The organic layers are separated, combined, dried with Na₂SO₄, filtered and concentrated in vacuo to afford the desired (3,4-dimethoxyphenyl)-(5-phenyl-3-sulfanyl-thiazolo[2,3-c][1,2,4]triazol-6-yl)methanone (0.30 g) which is used for next step without further purification.

Treatment of (3,4-dimethoxyphenyl)-(5-phenyl-3-sulfanyl-thiazolo[2,3-c][1,2,4]triazol-6-yl)methanone (0.3 g, 0.75 mmol) with 2-(bromomethyl)-1-chloro-3-fluoro-benzene (0.2 g, 0.9 mmol), Et₃N in dichloromethane at 0° C. using the similar alkylation procedure described above affords the desired title compound (0.24 g,

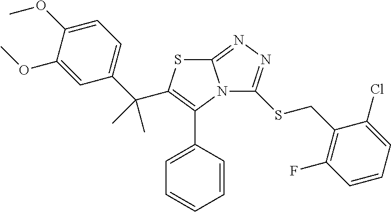 [SMILES: COc1ccc(C(C)(C)c2sc3nnc(SCc4c(F)cccc4Cl)n3c2-c2ccccc2)cc1OC]

### 3-[(2-chloro-6-fluoro-phenyl)methylsulfanyl]-6-[1-(3,4-dimethoxyphenyl)-1-methyl-ethyl]-5-phenyl-thiazolo[2,3-c][1,2,4]triazole

A solution of ZnMe₂ (2 M in toluene, 0.69 mL, 1.38 mmol) is added to a solution of TiCl₄ (1 M in toluene, 1.38 mL, 1.38 mmol) at −30° C. dropwise under N₂. The mixture is stirred at −30° C. for 20 min and added a solution of [3-[(2-chloro-6-fluoro-phenyl)methylsulfanyl]-5-phenyl-thiazolo[2,3-c][1,2,4]triazol-6-yl]-(3,4-dimethoxyphenyl)methanone (0.24 g, 0.46 mmol) in dichloromethane dropwise. The resulting mixture is stirred at −30° C. for additional 20 min and warmed up to room temperature, and is stirred for another 1.5 hr. The reaction mixture is slowly poured into a mixture of dry ice-MeOH and stirred for 2 hr at room temperature. The crude residue is taken up to EtOAc and washed with water. The organic layer is separated, dried with Na₂SO₄, filtered and concentrated in vacuo. The desired title compound is isolated by preparative HPLC (0.1 g, 40%) as a solid. LC/MS (m/z) M+H=554.5; t_{R}=3.22 min.

### Example 12

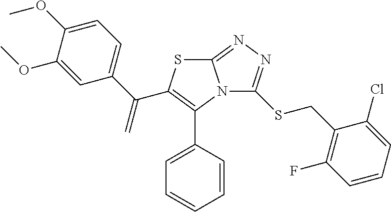 [SMILES: C=C(c1ccc(OC)c(OC)c1)c1sc2nnc(SCc3c(F)cccc3Cl)n2c1-c1ccccc1]

### 3-[(2-chloro-6-fluoro-phenyl)methylsulfanyl]-6-[1-(3,4-dimethoxyphenyl)vinyl]-5-phenyl-thiazolo[2,3-c][1,2,4]triazole

The title compound is isolated by preparative HPLC (2 mg, 0.8%) as a by-product from the preceding reaction of [3-[(2-chloro-6-fluoro-phenyl)methylsulfanyl]-5-phenyl-thiazolo[2,3-c][1,2,4]triazol-6-yl]-(3,4-dimethoxyphenyl)methanone with ZnMe₂ and TiCl₄. LCMS (m/z) M+H=538.4; t_{R}=3.17 min.

### Example 13

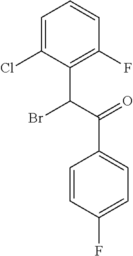 [SMILES: O=C(c1ccc(F)cc1)C(Br)c1c(F)cccc1Cl]

### 2-Bromo-2-(2-chloro-6-fluoro-phenyl)-1-(4-fluorophenyl)ethanone

Benzotriazole-1-yl-oxy-tris-(dimethylamino)-phosphonium hexafluorophosphate (3.51 g, 7.95 mmol) is added to a mixture of 2-(2-chloro-6-fluoro-phenyl)acetic acid (1.00 g, 5.30 mmol), N,O-dimethylhydroxylamine hydrochloride (1.03 g, 10.6 mmol) and triethylamine (3.69 mL, 26.5 mmol) in acetonitrile. The resulting mixture is stirred at 80° C. for 16 hr and concentrated in vacuo. The crude residue is taken up to EtOAc (100 mL) and washed with saturated NaHCO₃. The organic layer is separated, dried with Na₂SO₄, filtered and concentrated in vacuo to give crude product which is purified by chromatograph to give 2-(2-chloro-6-fluoro-phenyl)-N-methoxy-N-methyl-acetamide (1.17 g, 96%). LC/MS (m/z) M+H=232.3; t_{R}=2.4 min.

4-Fluoro-phenyl magnesium bromide (1 M in THF, 7.57 mL, 7.57 mmol) is added dropwise to a solution of 2-(2-chloro-6-fluoro-phenyl)-N-methoxy-N-methyl-acetamide (1.17 g, 5.05 mmol) in THF (25 mL) at −78° C. The resulting mixture is warmed up to room temperature and stirred for 1 hr at room temperature. The reaction mixture is quenched with saturated NH₄Cl at 0° C. and extracted with EtOAc. The organic layer is separated, dried with Na₂SO₄, filtered and concentrated in vacuo to give a crude product which is purified by chromatograph to give 2-(2-chloro-6-fluoro-phenyl)-1-(4-fluorophenyl)ethanone (0.10 g, 8%). A batch of this ketone is brominated with bromine in HOAc as described above to afford the title compound 2-Bromo-2-(2-chloro-6-fluoro-phenyl)-1-(4-fluorophenyl)ethanone (0.82 g, 99%).

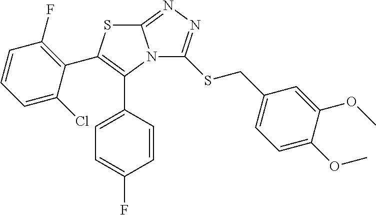 [SMILES: COc1ccc(CSc2nnc3sc(-c4c(F)cccc4Cl)c(-c4ccc(F)cc4)n23)cc1OC]

### 6-(2-Chloro-6-fluoro-phenyl)-3-[(3,4-dimethoxyphenyl)methylsulfanyl]-5-(4-fluorophenyl)thiazolo[2,3-c][1,2,4]triazole

Treatment of 2-bromo-2-(2-chloro-6-fluoro-phenyl)-1-(4-fluorophenyl)ethanone with hydrazinecarbothioamide followed by CS₂ using the same procedure descried above affords 6-(2-Chloro-6-fluoro-phenyl)-5-(4-fluorophenyl)thiazolo[2,3-c][1,2,4]triazole-3-thiol (0.69 g, 77%). LC/MS (m/z) M+H=380.3; t_{R}=2.86 min. This thiol intermediate is treated with (3,4-dimethoxyphenyl)methyl methanesulfonate, Et₃N in dichloromethane at 0° C. using the same alkylation procedure described above to afford the title compound (0.02 g, 25%). LC/MS (m/z) M+H=530.3; t_{R}=3.07 min.

### Example 14

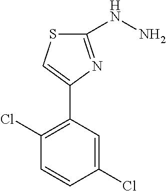 [SMILES: NNc1nc(-c2cc(Cl)ccc2Cl)cs1]

### 4-(2,5-dichlorophenyl)-2-hydrazinylthiazole

A mixture of 2-bromo-1-(2,5-dichlorophenyl)ethanone (1.00 g, 3.73 mmol) and hydrazinecarbothioamide (0.340 g, 3.73 mmol) in EtOH (50 mL) is heated at 80° C. for 12 hours. The reaction mixture is concentrated under reduced pressure to remove the solvent. The crude product is taken up in dichloromethane, washed with saturated NaHCO₃. The organic layer is separated, dried with Na₂SO₄, filtered and concentrated in vacuo to give title product as a yellow solid (0.95 g, 98% yield, purity >70%), which is used without further purification. LC/MS (m/z) M+H=260.3, 262.3; t_{R}=1.97 min.

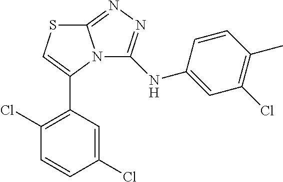 [SMILES: Cc1ccc(Nc2nnc3scc(-c4cc(Cl)ccc4Cl)n23)cc1Cl]

### N-(3-chloro-4-methylphenyl)-5-(2,5-dichlorophenyl)thiazolo[2,3-c][1,2,4]triazol-3-amine

2-Chloro-4-isothiocyanato-1-methylbenzene (0.200 g, 0.768 mmol) is added to a solution of 5-bromo-4-(2,5-dichlorophenyl)-2-hydrazinylthiazole (0.141 g, 0.768 mmol) in benzene (50 mL) and the mixture is heated at 80° C. for 1 hour. DCC (0.198 g, 0.961 mmol) is added to the mixture and heating at 80° C. is continued for 4 hours.

The mixture is concentrated under reduced pressure to remove the solvent. The crude product is absorbed onto silica gel and purified by silica column chromatography to afford the title compound as a white solid (0.097 g, 31%). LC/MS (m/z) M+H=409.3; t_{R}=2.97 min.

### Example 15

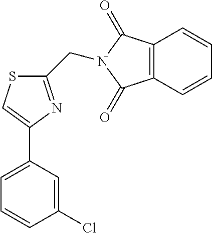 [SMILES: O=C1c2ccccc2C(=O)N1Cc1nc(-c2cccc(Cl)c2)cs1]

### 2-[[4-(3-chlorophenyl)thiazol-2-yl]methyl]isoindoline-1,3-dione

2-Bromo-1-(3-chlorophenyl)ethanone (0.28 g, 1.17 mmol) is added to 2-(1,3-dioxoisoindolin-2-yl)thioacetamide (0.20 g, 0.90 mmol) in EtOH (10 mL) and the mixture is heated to 80° C. for 90 min. The mixture is then cooled to room temperature which lead to the precipitation of the title compound 2-[[4-(3-chlorophenyl)thiazol-2-yl]methyl]isoindoline-1,3-dione (0.23 g, 70%). LC/MS (m/z) M+H=355.4; t_{R}=3.3 min.

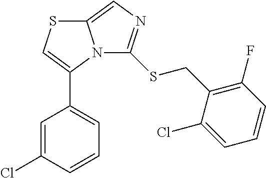 [SMILES: Fc1cccc(Cl)c1CSc1ncc2scc(-c3cccc(Cl)c3)n12]

### 5-[(2-chloro-6-fluoro-phenyl)methylsulfanyl]-3-(3-chlorophenyl)imidazo[5,1-b]thiazole

Ethylene diamine (0.051 mL, 0.76 mmol) is added to a mixture of 2-[[4-(3-chlorophenyl)thiazol-2-yl]methyl]isoindoline-1,3-dione (0.23 g, 0.63 mmol) in THF (6 mL). After stirring for overnight at room temperature the mixture is concentrated and washed through a short silica gel plug to give (4-(3-chlorophenyl)thiazol-2-yl)methanamine (90 mg, 63.2%), LC/MS (m/z) M+H=225.3; t_{R}=1.9 min. A portion of the amine (40 mg, 0.18 mmol) is subsequently treated with thiophosgene (0.014 mL, 0.18 mmol) and potassium carbonate (50 mg, 0.36 mmol) in a mixture of dichloromethane and water (1:1, 2 mL) at room temperature for 80 min. The reaction is then quenched with saturated NH₄Cl solution (15 mL) and washed with dichloromethane (3×10 mL). The combined organics are dried over Na₂SO₄ and concentrated to afford the crude 3-(3-chlorophenyl)-6H-imidazo[5,1-b]thiazole-5-thione. LC/MS (m/z) M+H=267.3; t_{R}=3.4 min.

To a mixture of the crude 3-(3-chlorophenyl)-6H-imidazo[5,1-b]thiazole-5-thione (48 mg, 0.18 mmol) in dichloromethane (1.0 mL) is added Hunig's base (0.094 mL, 0.54 mmol), followed by 2-(bromomethyl)-1-chloro-3-fluoro-benzene (0.037 mL, 0.27 mmol). The reaction mixture is stirred at room temperature for overnight, quenched with saturated NH₄Cl solution (20 mL), and washed with dichloromethane (3×10 mL). The combined organics are then dried over Na₂SO₄ and concentrated. The crude material is purified by preparative HPLC to give the title compound as a solid (6 mg, 8% yield). LC/MS (m/z) M+H=409.4; t_{R}=3.1 min.

### Example 16

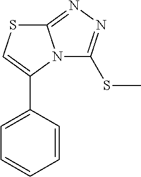 [SMILES: CSc1nnc2scc(-c3ccccc3)n12]

### 3-Methylsulfanyl-5-phenyl-thiazolo[2,3-c][1,2,4]triazole

A mixture of 5-phenylthiazolo[2,3-c][1,2,4]triazole-3-thiol (1.00 g, 4.29 mmol) and Cs₂CO₃ (2.79 g, 8.58 mmol) in acetone is stirred at room temperature for 10 min. To this solution is added MeI (0.91 g, 6.44 mmol) and the resulting mixture is stirred at room temperature for 3 hrs. The reaction mixture is concentrated under reduced pressure to remove the solvent. The crude product is taken up in dichloromethane and then washed with saturated NaHCO₃. The organic layer is separated, dried with Na₂SO₄, filtered and concentrated in vacuo to give crude product as a yellow solid. The crude product is absorbed onto silica gel and purified by column chromatography using a gradient of Hexane (100%) to EtOAc (100%). The title product is collected as a white solid (1.00 g, 94%). LC/MS (m/z) M+H=248.3; t_{R}=2.31 min.

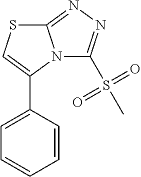 [SMILES: CS(=O)(=O)c1nnc2scc(-c3ccccc3)n12]

### 3-Methylsulfonyl-5-phenyl-thiazolo[2,3-c][1,2,4]triazole

mCPBA (3.70 g, 10.73 mmol) is added to a solution of 3-methylsulfanyl-5-phenyl-thiazolo[2,3-c][1,2,4]triazole (1.00 g, 4.29 mmol) in dichloromethane at 0° C. The resulting mixture is stirred at room temperature for 18 hrs. The resulting mixture is washed with saturated NaHCO₃. The organic layer is separated, dried with Na₂SO₄, filtered and concentrated in vacuo to give the title compound as a yellow solid. This crude product is used without further purification. LC/MS (m/z) M+H=280.3; t_{R}=2.15 min.

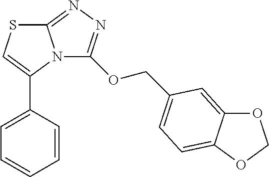 [SMILES: c1ccc(-c2csc3nnc(OCc4ccc5c(c4)OCO5)n23)cc1]

### 3-(1,3-Benzodioxol-5-ylmethoxy)-5-phenyl-thiazolo[2,3-c][1,2,4]triazole

A mixture of 3-Methylsulfonyl-5-phenyl-thiazolo[2,3-c][1,2,4]triazole (0.02 g, 0.06 mmol), piperonyl alcohol (0.01 g, 0.10 mmol) and Cs₂CO₃ (0.05 g, 0.16 mmol) in DMPU (0.8 mL) is stirred at 200° C. for 80 min in microwave. The crude product is taken up in dichloromethane and washed with saturated NaHCO₃. The organic layer is separated, dried with Na₂SO₄, filtered and concentrated in vacuo to give crude product as a yellow solid. The crude product is purified by preparative HPLC to give the title compound (0.01 g, 47%) as a white solid. LC/MS (m/z) M+H=352.4; t_{R}=3.14 min.

### Example 17

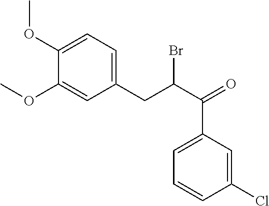 [SMILES: COc1ccc(CC(Br)C(=O)c2cccc(Cl)c2)cc1OC]

### 2-Bromo-1-(3-chlorophenyl)-3-(3,4-dimethoxyphenyl)propan-1-one

A mixture of 3-(3,4-dimethoxyphenyl)propanoic acid (2.00 g, 8.69 mmol), N,O-dimethylhydroxylamine hydrochloride (1.27 g, 13.03 mmol), DMAP (1.59 g, 13.03 mmol) and EDC (2.21 g, 11.55 mmol) in dichloromethane is stirred at room temperature for 12 hrs and quenched with 1 N HCl. The mixture is extracted with dichloromethane and washed with saturated NaHCO₃. The organic layer is separated, dried with Na₂SO₄, filtered and concentrated in vacuo to give desired 3-(3,4-dimethoxyphenyl)-N-methoxy-N-methyl-propanamide (2.0 g, 88%) as a white solid, which is used without further purification. TLC: R_{f}=0.71 (dichloromethane/EtOAc/MOH; 1:1:0.1)

A solution of 3-chloro-phenyl magnesium bromide (7.91 mL, 7.91 mmol) in THF is added to a solution of 3-(3,4-dimethoxyphenyl)-N-methoxy-N-methyl-propanamide (2.0 g, 7.91 mmol) in THF at −78° C. The mixture is stirred at 0° C. for 2 hours, quenched by saturated NH₄Cl at 0° C. and concentrated under reduced pressure to remove the solvent. The crude product is taken in by dichloromethane and washed with saturated NaHCO₃. The organic layer is separated, dried with Na₂SO₄, filtered and concentrated in vacuo. The yellow crude product is absorbed onto silica gel and purified by column chromatography to afford 1-(3-Chlorophenyl)-3-(3,4-dimethoxyphenyl)propan-1-one (2.0 g, 85%). TLC: R_{f}=0.71 (Hexane/EtOAc; 1:1)

n-Bu₄NBr₃ (0.63 g, 1.31 mmol) is added to a solution of 1-(3-chlorophenyl)-3-(3,4-dimethoxyphenyl)propan-1-one (0.40 g, 1.31 mmol) in dichloromethane at 0° C. and the resulting mixture is stirred at 0° C. for 30 min. The reaction mixture is quenched with water and extracted with dichloromethane. The organic layer is separated, dried with Na₂SO₄, filtered and concentrated in vacuo to give desired 2-bromo-1-(3-chlorophenyl)-3-(3,4-dimethoxyphenyl)propan-1-one (0.45 g, 90%), which is used without further purification. TLC: R_{f}=0.77 (Hexane/EtOAc; 1:1)

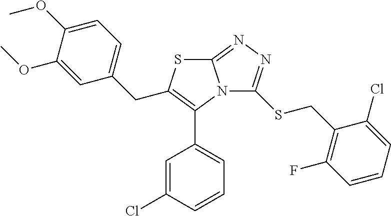 [SMILES: COc1ccc(Cc2sc3nnc(SCc4c(F)cccc4Cl)n3c2-c2cccc(Cl)c2)cc1OC]

### 3-[(2-Chloro-6-fluoro-phenyl)methylsulfanyl]-5-(3-chlorophenyl)-6-[(3,4-dimethoxyphenyl)methyl]thiazolo[2,3-c][1,2,4]triazole

Treatment of 2-bromo-1-(3-chlorophenyl)-3-(3,4-dimethoxyphenyl)propan-1-one with hydrazinecarbothioamide followed by CS₂ as previously described affords 5-(3-chlorophenyl)-6-[(3,4-dimethoxyphenyl)methyl]thiazolo[2,3-c][1,2,4]triazole-3-thiol (0.30 g, 45%). LC/MS (m/z) M+H=418.4; t_{R}=2.82 min.

A mixture of 5-(3-chlorophenyl)-6-[(3,4-dimethoxyphenyl)methyl]thiazolo[2,3-c][1,2,4]triazole-3-thiol (0.03 g, 0.07 mmol), 2-(bromomethyl)-1-chloro-3-fluoro-benzene (0.02 g, 0.09 mmol) and K₂CO₃ (0.02 g, 0.14 mmol) in acetone (2 mL) is stirred at room temperature for 2 hr. Water (2 mL) is added, and the reaction mixture is extracted with dichloromethane. The organic layer is separated, dried with Na₂SO₄, filtered and concentrated in vacuo. The crude is purified by preparative HPLC to give desired title compound (8.0 mg, 20%) as solid. LC/MS (m/z) M+H=560.4; t_{R}=3.36 min.

### Example 18

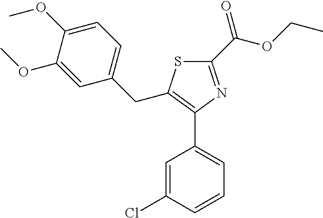 [SMILES: CCOC(=O)c1nc(-c2cccc(Cl)c2)c(Cc2ccc(OC)c(OC)c2)s1]

### ethyl 4-(3-chlorophenyl)-5-[(3,4-dimethoxyphenyl)methyl]thiazole-2-carboxylate

Ethyl thiooxamate (0.67 g, 5.0 mmol) is added to 2-bromo-1-(3-chlorophenyl)-3-(3,4-dimethoxyphenyl)propan-1-one (1.92 g, 5.0 mmol) in EtOH (40 mL). The reaction mixture is then refluxed for 2 hours, concentrated and the residue purified by chromatography to give the title compound (0.80 g, 38%). LC/MS (m/z) M+H=418.4; t_{R}=3.4 min.

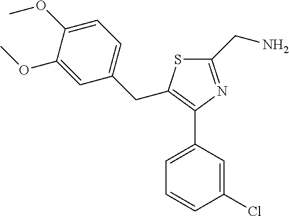 [SMILES: COc1ccc(Cc2sc(CN)nc2-c2cccc(Cl)c2)cc1OC]

### [4-(3-chlorophenyl)-5-[(3,4-dimethoxyphenyl)methyl]thiazol-2-yl]methanamine

Diisobutylaluminum hydride (6.3 mL, 1 M solution in dichloromethane) is added to ethyl 4-(3-chlorophenyl)-5-(3,4-dimethoxybenzyl)thiazole-2-carboxylate (0.80 g, 1.9 mmol) in THF (10 mL) at 0° C. After 10 min the reaction is allowed to warm to room temperature over 3 hr. The mixture is quenched with dropwise of 3 mL MeOH, and Et₂O (100 mL) is added followed by 15% Rochelle's salt solution (100 mL). Vigorous stirring is continued until the solution becomes clear (˜3 hr). The organic layer is set aside and the aqueous layer is washed with Et₂O (2×75 mL). The combined organics are washed with brine, dried over sodium sulfate, concentrated and the resulting oil is azeotroped from toluene (2×). LC/MS (m/z) M+H=376.5; t_{R}=2.9 min. To the crude primary alcohol (0.58 g, 1.5 mmol) is added diethyl azodicarboxylate (0.61 mL, 3.1 mmol), PPh₃ (0.81 g, 3.1 mmol) and phthalimide (0.34 g, 2.3 mmol) in THF (10 mL) at 0° C. After 10 min the ice bath is removed and stirring continued for 2 hr. The mixture is concentrated and purified via chromatography to give the desired phthalimide derivative (0.34 g, 43%). LC/MS (m/z) M+H=505.5; t_{R}=3.4 min. Ethylene diamine (0.18 mL, 2.7 mmol) is added to the phthalimide (0.34 g, 0.7 mmol) in a mixture of 1:1 THF-MeOH (6 mL). Stirring is continued for 4 hr, the mixture is concentrated and purified via chromatography to give [4-(3-chlorophenyl)-5-[(3,4-dimethoxyphenyl)methyl]thiazol-2-yl]methanamine (0.20 g, 80%). LC/MS (m/z) M+H=375.4; t_{R}=2.3 min.

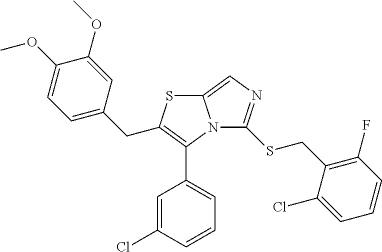 [SMILES: COc1ccc(Cc2sc3cnc(SCc4c(F)cccc4Cl)n3c2-c2cccc(Cl)c2)cc1OC]

### 5-[(2-chloro-6-fluoro-phenyl)methylsulfanyl]-3-(3-chlorophenyl)-2-[(3,4-dimethoxyphenyl)methyl]imidazo[5,1-b]thiazole

Thiophosgene (0.014 mL, 0.19 mmol) is added to [4-(3-chlorophenyl)-5-[(3,4-dimethoxyphenyl)methyl]thiazol-2-yl]methanamine (0.07 g, 0.19) and potassium carbonate (0.05 g, 0.37 mmol) in dichloromethane-water (1:1, 4 mL) at room temperature. Stirring is continued for 3 hr, the mixture is diluted with EtOAc and washed with ammonium chloride solution (2×) and brine. The organic layer is dried over sodium sulfate and concentrated. LC/MS (m/z) M+H=417.4; t_{R}=3.7 min. To a mixture of the crude thiol (0.03 g, 0.06 mmol) and triethylamine (0.025 mL, 0.18 mmol) in dichloromethane (2 mL) is added 2-(bromomethyl)-1-chloro-3-fluorobenzene (0.013 mL, 0.09 mmol), Stirring is continued for 2 hr, the mixture is diluted with EtOAc (5 mL) and washed with ammonium chloride solution (2×) and brine. The organic layer is dried over sodium sulfate and concentrated. The crude is purified by chromatography to afford the title compound (14 mg, 40%) as a solid. LC/MS (m/z) M+H=559.5; t_{R}=3.4 min.

### Examples 19-137

The following compounds are prepared essentially according to the procedures set forth above, with modifications where necessary of the starting materials to provide the desired product.

| Example | | Measured |
|---|---|---|
| No. | Names | M + H |
| 19 | 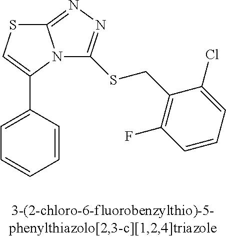 [SMILES: Fc1cccc(Cl)c1CSc1nnc2scc(-c3ccccc3)n12] | 376.3 |
| 20 | 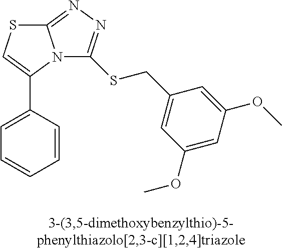 [SMILES: COc1cc(CSc2nnc3scc(-c4ccccc4)n23)cc(OC)c1] | 384.4 |
| 21 | 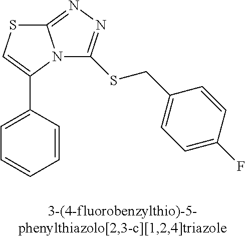 [SMILES: Fc1ccc(CSc2nnc3scc(-c4ccccc4)n23)cc1] | 342.4 |
| 22 | 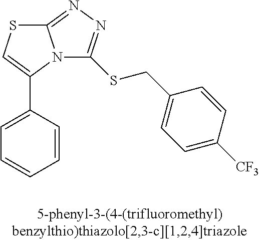 [SMILES: FC(F)(F)c1ccc(CSc2nnc3scc(-c4ccccc4)n23)cc1] | 392.3 |
| 23 | 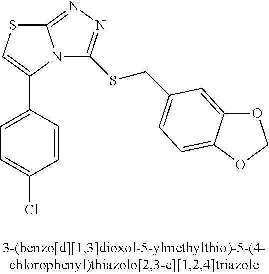 [SMILES: Clc1ccc(-c2csc3nnc(SCc4ccc5c(c4)OCO5)n23)cc1] | 402.3 |
| 24 | 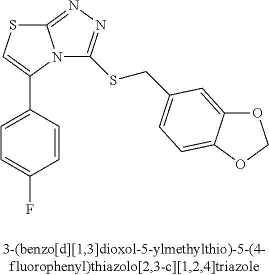 [SMILES: Fc1ccc(-c2csc3nnc(SCc4ccc5c(c4)OCO5)n23)cc1] | 386.3 |
| 25 | 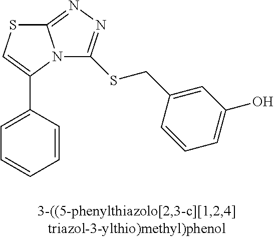 [SMILES: Oc1cccc(CSc2nnc3scc(-c4ccccc4)n23)c1] | 340.3 |
| 26 | 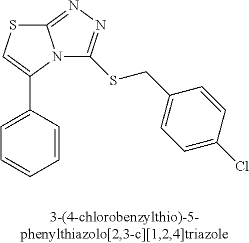 [SMILES: Clc1ccc(CSc2nnc3scc(-c4ccccc4)n23)cc1] | 358.3 |
| 27 | 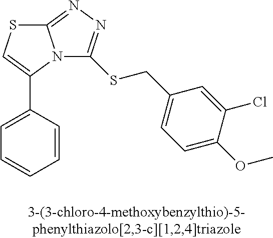 [SMILES: COc1ccc(CSc2nnc3scc(-c4ccccc4)n23)cc1Cl] | 388.3 |
| 28 | 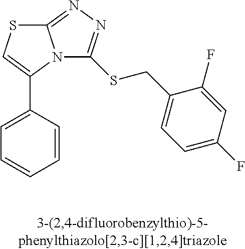 [SMILES: Fc1ccc(CSc2nnc3scc(-c4ccccc4)n23)c(F)c1] | 360.3 |
| 29 | 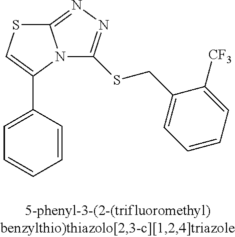 [SMILES: FC(F)(F)c1ccccc1CSc1nnc2scc(-c3ccccc3)n12] | 392.3 |
| 30 | 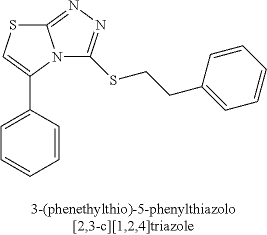 [SMILES: c1ccc(CCSc2nnc3scc(-c4ccccc4)n23)cc1] | 338.4 |
| 31 | 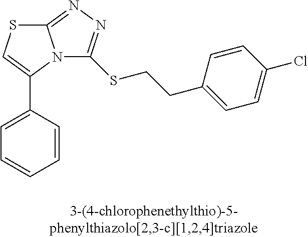 [SMILES: Clc1ccc(CCSc2nnc3scc(-c4ccccc4)n23)cc1] | 372.3 |
| 32 | 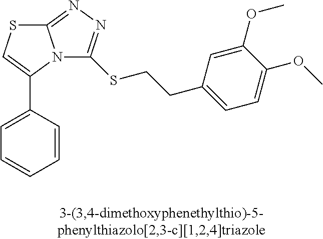 [SMILES: COc1ccc(CCSc2nnc3scc(-c4ccccc4)n23)cc1OC] | 398.4 |
| 33 | 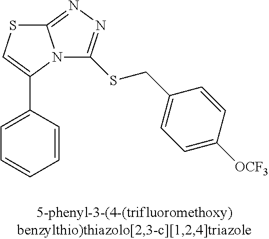 [SMILES: FC(F)(F)Oc1ccc(CSc2nnc3scc(-c4ccccc4)n23)cc1] | 408.3 |
| 34 | 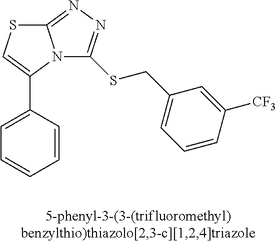 [SMILES: FC(F)(F)c1cccc(CSc2nnc3scc(-c4ccccc4)n23)c1] | 392.3 |
| 35 | 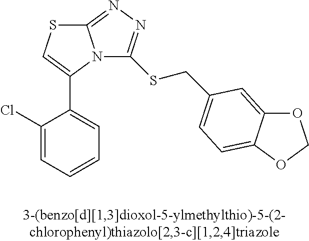 [SMILES: Clc1ccccc1-c1csc2nnc(SCc3ccc4c(c3)OCO4)n12] | 402.3 |
| 36 | 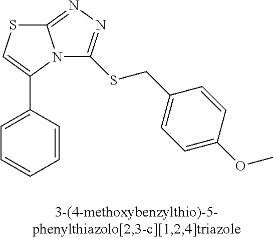 [SMILES: COc1ccc(CSc2nnc3scc(-c4ccccc4)n23)cc1] | 354.4 |
| 37 | 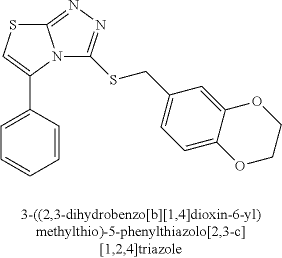 [SMILES: c1ccc(-c2csc3nnc(SCc4ccc5c(c4)OCCO5)n23)cc1] | 382.4 |
| 38 | 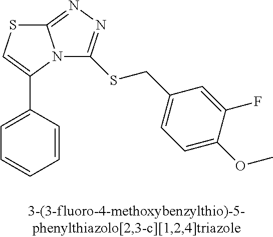 [SMILES: COc1ccc(CSc2nnc3scc(-c4ccccc4)n23)cc1F] | 372.4 |
| 39 | 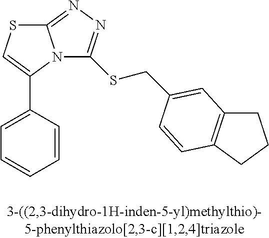 [SMILES: c1ccc(-c2csc3nnc(SCc4ccc5c(c4)CCC5)n23)cc1] | 364.4 |
| 40 | 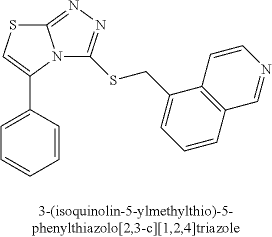 [SMILES: c1ccc(-c2csc3nnc(SCc4cccc5cnccc45)n23)cc1] | 375.4 |
| 41 | 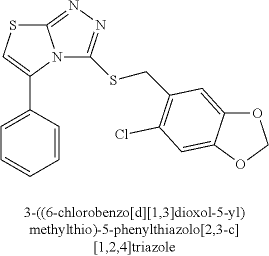 [SMILES: Clc1cc2c(cc1CSc1nnc3scc(-c4ccccc4)n13)OCO2] | 402.3 |
| 42 | 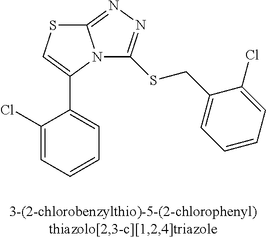 [SMILES: Clc1ccccc1CSc1nnc2scc(-c3ccccc3Cl)n12] | 392.3 |
| 43 | 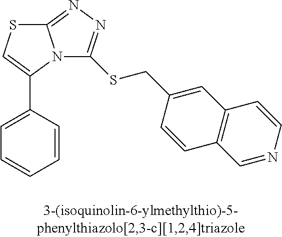 [SMILES: c1ccc(-c2csc3nnc(SCc4ccc5cnccc5c4)n23)cc1] | 375.4 |
| 44 | 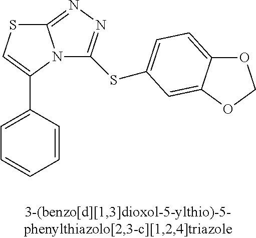 [SMILES: c1ccc(-c2csc3nnc(Sc4ccc5c(c4)OCO5)n23)cc1] | 354.3 |
| 45 | 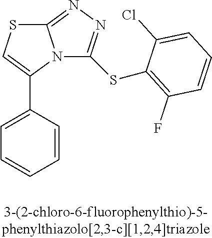 [SMILES: Fc1cccc(Cl)c1Sc1nnc2scc(-c3ccccc3)n12] | 362.3 |
| 46 | 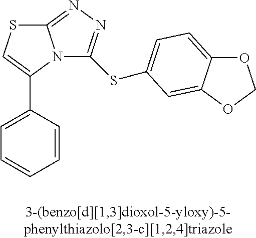 [SMILES: c1ccc(-c2csc3nnc(Sc4ccc5c(c4)OCO5)n23)cc1] | 338.4 |
| 47 | 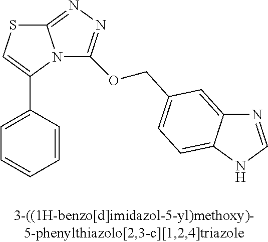 [SMILES: c1ccc(-c2csc3nnc(OCc4ccc5[nH]cnc5c4)n23)cc1] | 348.4 |
| 48 | 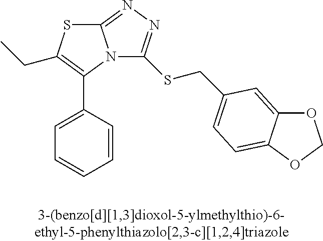 [SMILES: CCc1sc2nnc(SCc3ccc4c(c3)OCO4)n2c1-c1ccccc1] | 396.4 |
| 49 | 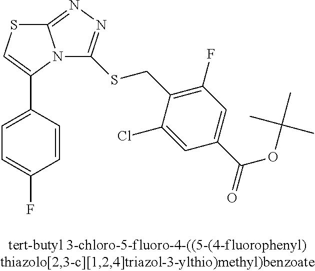 [SMILES: CC(C)(C)OC(=O)c1cc(F)c(CSc2nnc3scc(-c4ccc(F)cc4)n23)c(Cl)c1] | 494.3 |
| 50 | 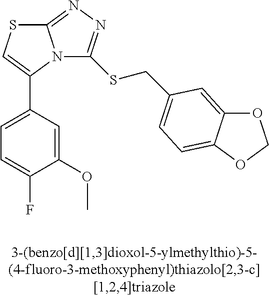 [SMILES: COc1cc(-c2csc3nnc(SCc4ccc5c(c4)OCO5)n23)ccc1F] | 416.4 |
| 51 | 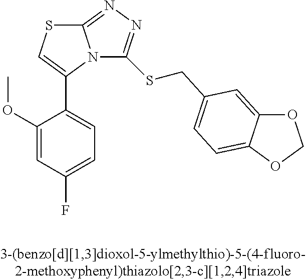 [SMILES: COc1cc(F)ccc1-c1csc2nnc(SCc3ccc4c(c3)OCO4)n12] | 416.4 |
| 52 | 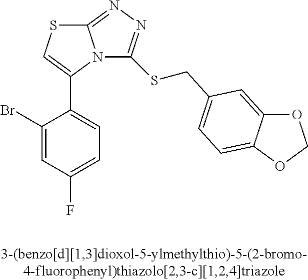 [SMILES: Fc1ccc(-c2csc3nnc(SCc4ccc5c(c4)OCO5)n23)c(Br)c1] | 464.3 |
| 53 | 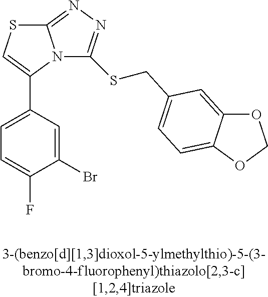 [SMILES: Fc1ccc(-c2csc3nnc(SCc4ccc5c(c4)OCO5)n23)cc1Br] | 464.3 |
| 54 | 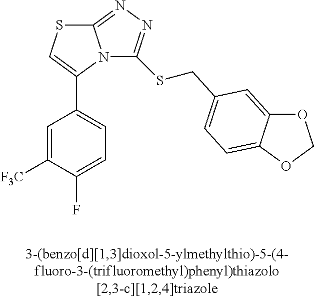 [SMILES: Fc1ccc(-c2csc3nnc(SCc4ccc5c(c4)OCO5)n23)cc1C(F)(F)F] | 454.3 |
| 55 | 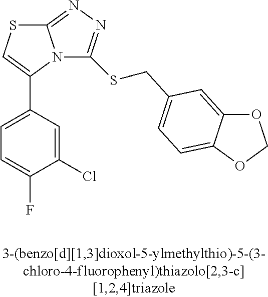 [SMILES: Fc1ccc(-c2csc3nnc(SCc4ccc5c(c4)OCO5)n23)cc1Cl] | 420.3 |
| 56 | 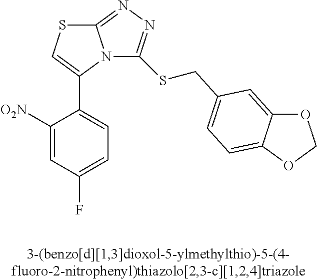 [SMILES: O=[N+]([O-])c1cc(F)ccc1-c1csc2nnc(SCc3ccc4c(c3)OCO4)n12] | 431.3 |
| 57 | 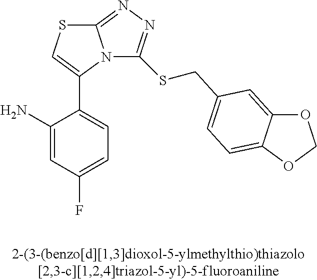 [SMILES: Nc1cc(F)ccc1-c1csc2nnc(SCc3ccc4c(c3)OCO4)n12] | 401.3 |
| 58 | 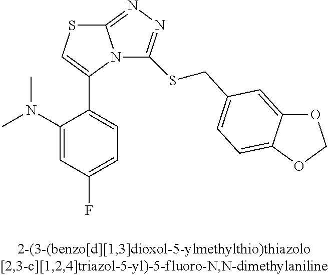 [SMILES: CN(C)c1cc(F)ccc1-c1csc2nnc(SCc3ccc4c(c3)OCO4)n12] | 429.4 |
| 59 | 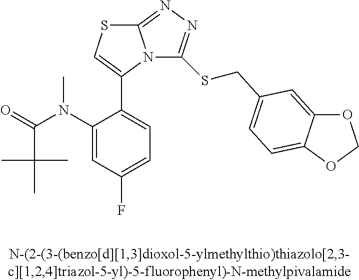 [SMILES: CN(C(=O)C(C)(C)C)c1cc(F)ccc1-c1csc2nnc(SCc3ccc4c(c3)OCO4)n12] | 499.4 |
| 60 | 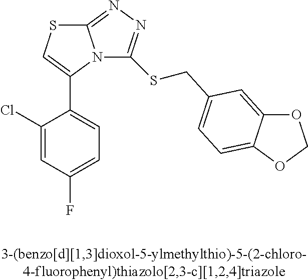 [SMILES: Fc1ccc(-c2csc3nnc(SCc4ccc5c(c4)OCO5)n23)c(Cl)c1] | 420.3 |
| 61 | 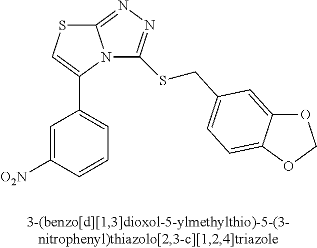 [SMILES: O=[N+]([O-])c1cccc(-c2csc3nnc(SCc4ccc5c(c4)OCO5)n23)c1] | 413.3 |
| 62 | 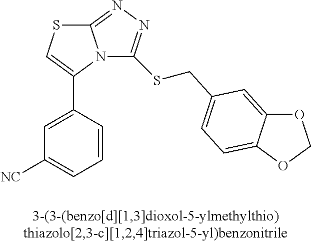 [SMILES: N#Cc1cccc(-c2csc3nnc(SCc4ccc5c(c4)OCO5)n23)c1] | 393.3 |
| 63 | 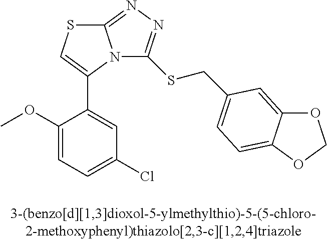 [SMILES: COc1ccc(Cl)cc1-c1csc2nnc(SCc3ccc4c(c3)OCO4)n12] | 432.3 |
| 64 | 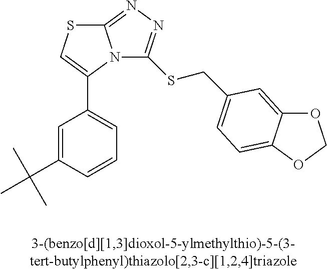 [SMILES: CC(C)(C)c1cccc(-c2csc3nnc(SCc4ccc5c(c4)OCO5)n23)c1] | 424.4 |
| 65 | 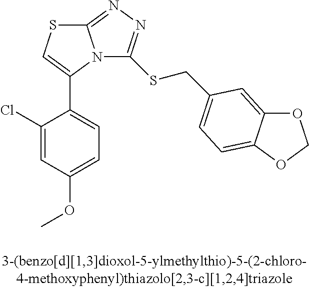 [SMILES: COc1ccc(-c2csc3nnc(SCc4ccc5c(c4)OCO5)n23)c(Cl)c1] | 432.3 |
| 66 | 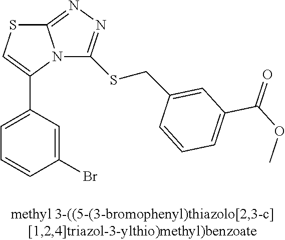 [SMILES: COC(=O)c1cccc(CSc2nnc3scc(-c4cccc(Br)c4)n23)c1] | 460.3 |
| 67 | 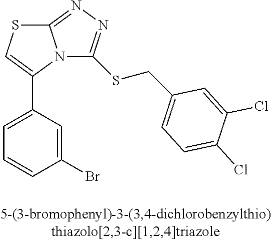 [SMILES: Clc1ccc(CSc2nnc3scc(-c4cccc(Br)c4)n23)cc1Cl] | 470.2 |
| 68 | 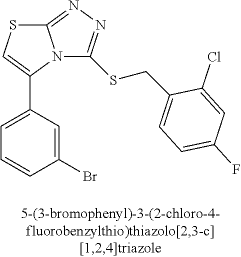 [SMILES: Fc1ccc(CSc2nnc3scc(-c4cccc(Br)c4)n23)c(Cl)c1] | 454.3 |
| 69 | 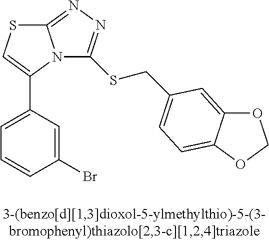 [SMILES: Brc1cccc(-c2csc3nnc(SCc4ccc5c(c4)OCO5)n23)c1] | 446.3 |
| 70 | 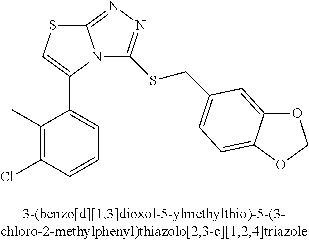 [SMILES: Cc1c(Cl)cccc1-c1csc2nnc(SCc3ccc4c(c3)OCO4)n12] | 416.3 |
| 71 | 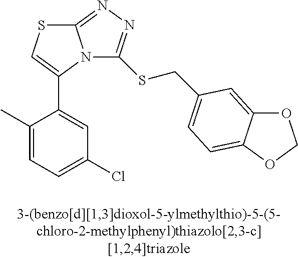 [SMILES: Cc1ccc(Cl)cc1-c1csc2nnc(SCc3ccc4c(c3)OCO4)n12] | 416.3 |
| 72 | 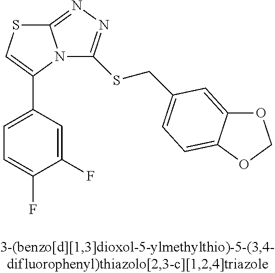 [SMILES: Fc1ccc(-c2csc3nnc(SCc4ccc5c(c4)OCO5)n23)cc1F] | 404.3 |
| 73 | 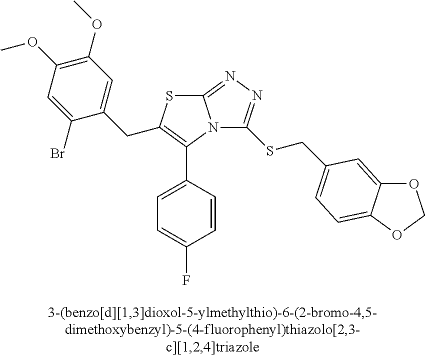 [SMILES: COc1cc(Br)c(Cc2sc3nnc(SCc4ccc5c(c4)OCO5)n3c2-c2ccc(F)cc2)cc1OC] | 614.3 |
| 74 | 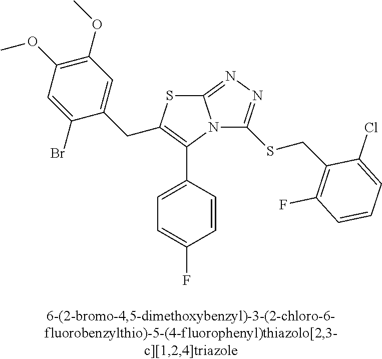 [SMILES: COc1cc(Br)c(Cc2sc3nnc(SCc4c(F)cccc4Cl)n3c2-c2ccc(F)cc2)cc1OC] | 622.3 |
| 75 | 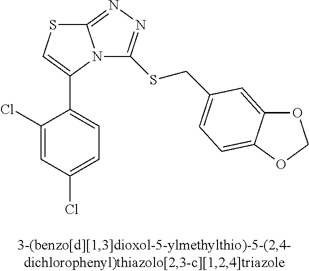 [SMILES: Clc1ccc(-c2csc3nnc(SCc4ccc5c(c4)OCO5)n23)c(Cl)c1] | 436.3 |
| 76 | 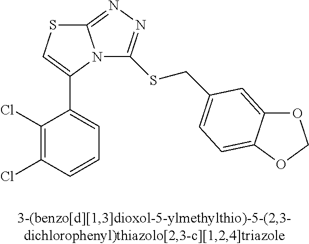 [SMILES: Clc1cccc(-c2csc3nnc(SCc4ccc5c(c4)OCO5)n23)c1Cl] | 436.3 |
| 77 | 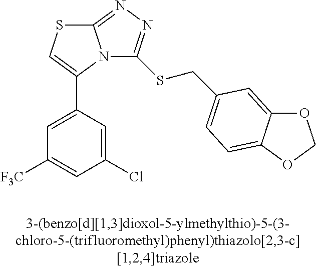 [SMILES: FC(F)(F)c1cc(Cl)cc(-c2csc3nnc(SCc4ccc5c(c4)OCO5)n23)c1] | 470.3 |
| 78 | 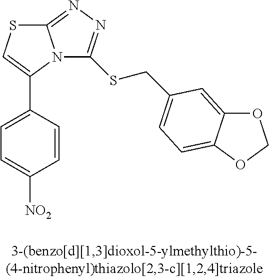 [SMILES: O=[N+]([O-])c1ccc(-c2csc3nnc(SCc4ccc5c(c4)OCO5)n23)cc1] | 413.3 |
| 79 | 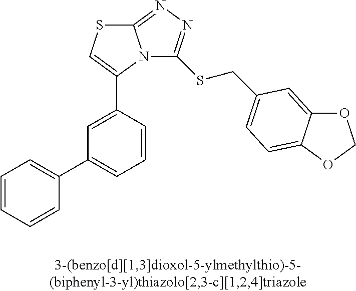 [SMILES: c1ccc(-c2cccc(-c3csc4nnc(SCc5ccc6c(c5)OCO6)n34)c2)cc1] | 444.4 |
| 80 | 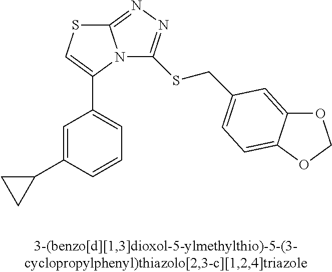 [SMILES: c1cc(-c2csc3nnc(SCc4ccc5c(c4)OCO5)n23)cc(C2CC2)c1] | 408.4 |
| 81 | 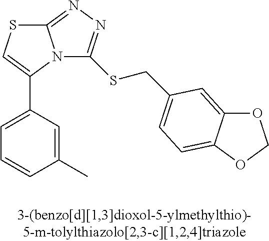 [SMILES: Cc1cccc(-c2csc3nnc(SCc4ccc5c(c4)OCO5)n23)c1] | 382.4 |
| 82 | 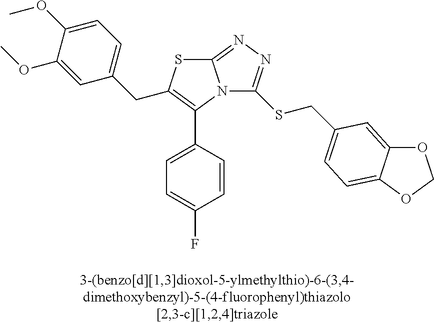 [SMILES: COc1ccc(Cc2sc3nnc(SCc4ccc5c(c4)OCO5)n3c2-c2ccc(F)cc2)cc1OC] | 536.4 |
| 83 | 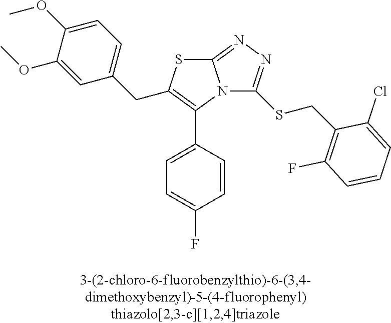 [SMILES: COc1ccc(Cc2sc3nnc(SCc4c(F)cccc4Cl)n3c2-c2ccc(F)cc2)cc1OC] | 544.4 |
| 84 | 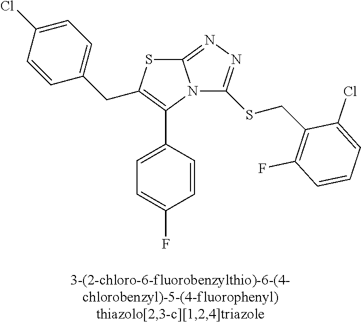 [SMILES: Fc1ccc(-c2c(Cc3ccc(Cl)cc3)sc3nnc(SCc4c(F)cccc4Cl)n23)cc1] | 518.3 |
| 85 | 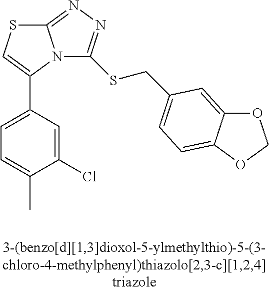 [SMILES: Cc1ccc(-c2csc3nnc(SCc4ccc5c(c4)OCO5)n23)cc1Cl] | 416.3 |
| 86 | 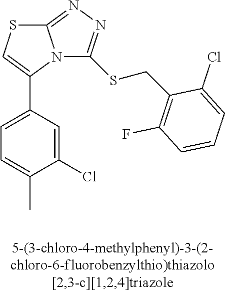 [SMILES: Cc1ccc(-c2csc3nnc(SCc4c(F)cccc4Cl)n23)cc1Cl] | 424.3 |
| 87 | 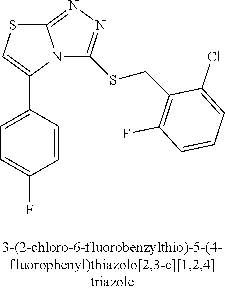 [SMILES: Fc1ccc(-c2csc3nnc(SCc4c(F)cccc4Cl)n23)cc1] | 394.3 |
| 88 | 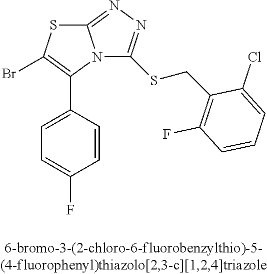 [SMILES: Fc1ccc(-c2c(Br)sc3nnc(SCc4c(F)cccc4Cl)n23)cc1] | 472.2 |
| 89 | 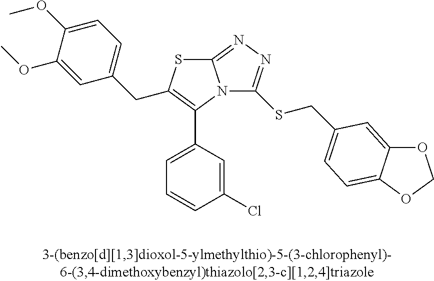 [SMILES: COc1ccc(Cc2sc3nnc(SCc4ccc5c(c4)OCO5)n3c2-c2cccc(Cl)c2)cc1OC] | 552.4 |
| 90 | 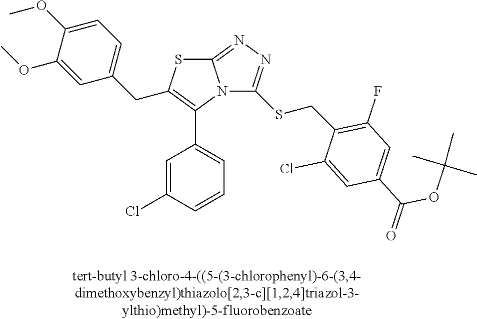 [SMILES: COc1ccc(Cc2sc3nnc(SCc4c(F)cc(C(=O)OC(C)(C)C)cc4Cl)n3c2-c2cccc(Cl)c2)cc1OC] | 660.4 |
| 91 | 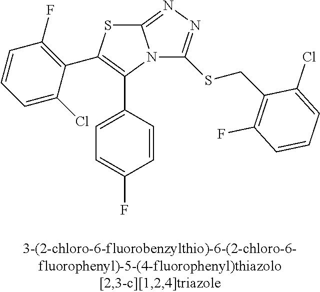 [SMILES: Fc1ccc(-c2c(-c3c(F)cccc3Cl)sc3nnc(SCc4c(F)cccc4Cl)n23)cc1] | 522.3 |
| 92 | 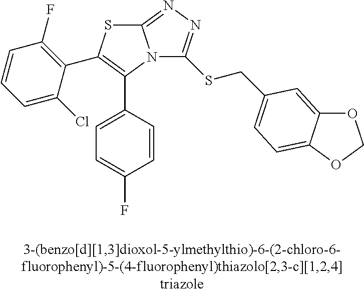 [SMILES: Fc1ccc(-c2c(-c3c(F)cccc3Cl)sc3nnc(SCc4ccc5c(c4)OCO5)n23)cc1] | 514.3 |
| 93 | 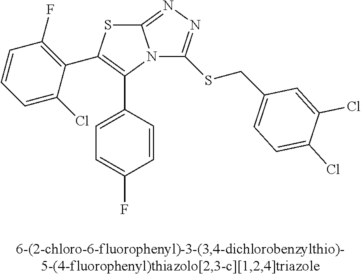 [SMILES: Fc1ccc(-c2c(-c3c(F)cccc3Cl)sc3nnc(SCc4ccc(Cl)c(Cl)c4)n23)cc1] | 538.3 |
| 94 | 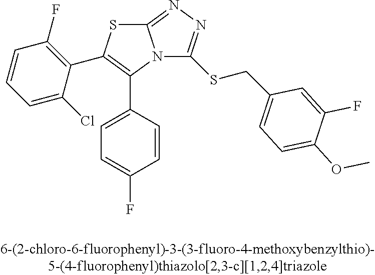 [SMILES: COc1ccc(CSc2nnc3sc(-c4c(F)cccc4Cl)c(-c4ccc(F)cc4)n23)cc1F] | 518.3 |
| 95 | 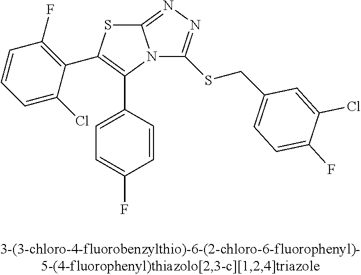 [SMILES: Fc1ccc(-c2c(-c3c(F)cccc3Cl)sc3nnc(SCc4ccc(F)c(Cl)c4)n23)cc1] | 522.3 |
| 96 | 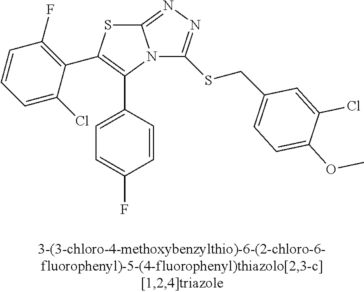 [SMILES: COc1ccc(CSc2nnc3sc(-c4c(F)cccc4Cl)c(-c4ccc(F)cc4)n23)cc1Cl] | 534.3 |
| 97 | 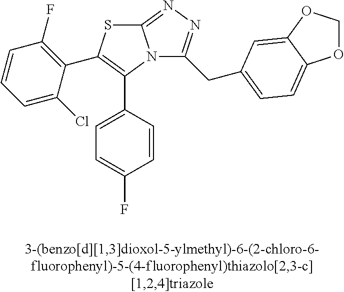 [SMILES: Fc1ccc(-c2c(-c3c(F)cccc3Cl)sc3nnc(Cc4ccc5c(c4)OCO5)n23)cc1] | 482.4 |
| 98 | 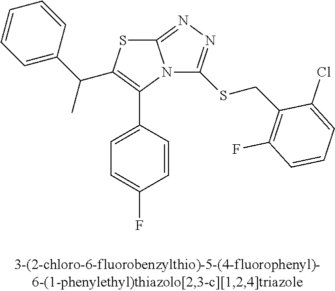 [SMILES: CC(c1ccccc1)c1sc2nnc(SCc3c(F)cccc3Cl)n2c1-c1ccc(F)cc1] | 498.4 |
| 99 | 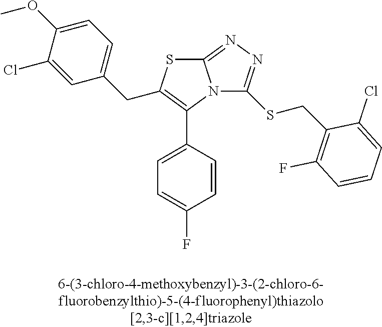 [SMILES: COc1ccc(Cc2sc3nnc(SCc4c(F)cccc4Cl)n3c2-c2ccc(F)cc2)cc1Cl] | 548.3 |
| 100 | 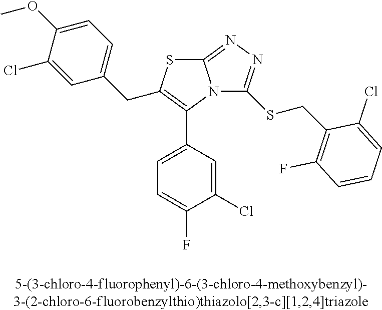 [SMILES: COc1ccc(Cc2sc3nnc(SCc4c(F)cccc4Cl)n3c2-c2ccc(F)c(Cl)c2)cc1Cl] | 582.3 |
| 101 | 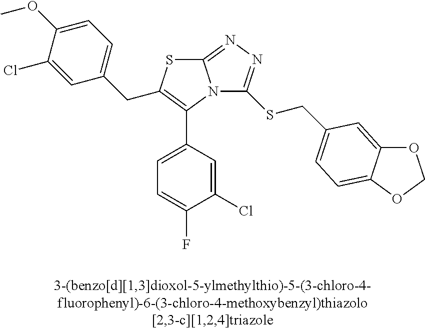 [SMILES: COc1ccc(Cc2sc3nnc(SCc4ccc5c(c4)OCO5)n3c2-c2ccc(F)c(Cl)c2)cc1Cl] | 574.3 |
| 102 | 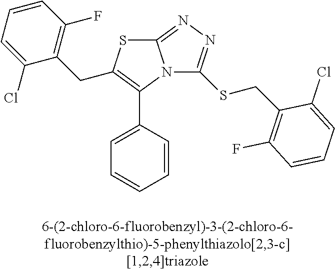 [SMILES: Fc1cccc(Cl)c1CSc1nnc2sc(Cc3c(F)cccc3Cl)c(-c3ccccc3)n12] | 518.3 |
| 103 | 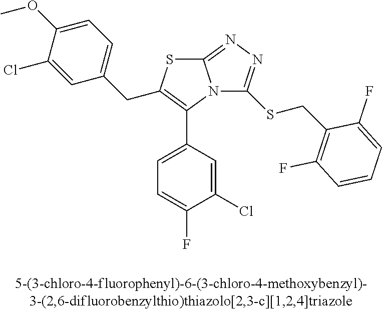 [SMILES: COc1ccc(Cc2sc3nnc(SCc4c(F)cccc4F)n3c2-c2ccc(F)c(Cl)c2)cc1Cl] | 566.3 |
| 104 | 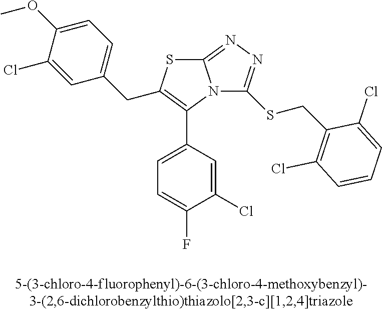 [SMILES: COc1ccc(Cc2sc3nnc(SCc4c(Cl)cccc4Cl)n3c2-c2ccc(F)c(Cl)c2)cc1Cl] | 598.3 |
| 105 | 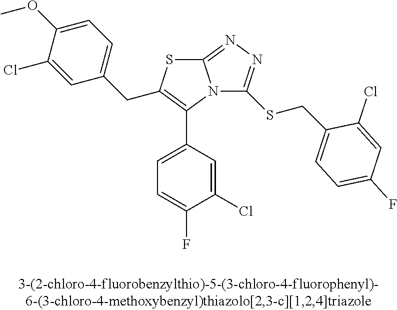 [SMILES: COc1ccc(Cc2sc3nnc(SCc4ccc(F)cc4Cl)n3c2-c2ccc(F)c(Cl)c2)cc1Cl] | 582.3 |
| 106 | 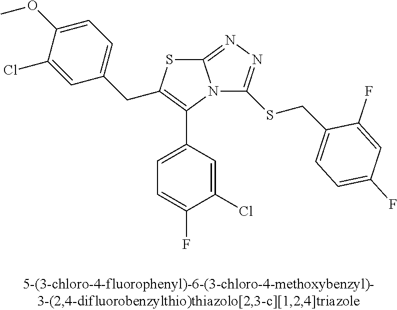 [SMILES: COc1ccc(Cc2sc3nnc(SCc4ccc(F)cc4F)n3c2-c2ccc(F)c(Cl)c2)cc1Cl] | 566.3 |
| 107 | 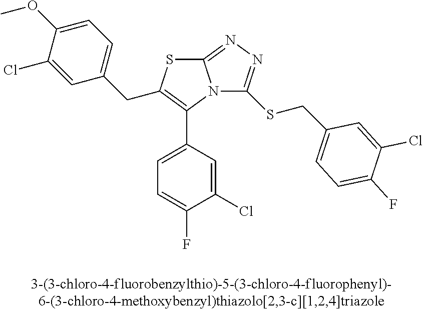 [SMILES: COc1ccc(Cc2sc3nnc(SCc4ccc(F)c(Cl)c4)n3c2-c2ccc(F)c(Cl)c2)cc1Cl] | 582.3 |
| 108 | 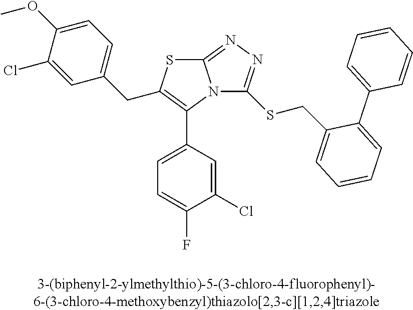 [SMILES: COc1ccc(Cc2sc3nnc(SCc4ccccc4-c4ccccc4)n3c2-c2ccc(F)c(Cl)c2)cc1Cl] | 606.4 |
| 109 | 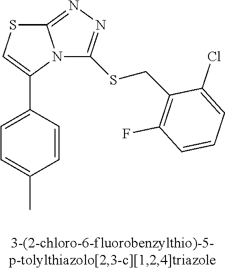 [SMILES: Cc1ccc(-c2csc3nnc(SCc4c(F)cccc4Cl)n23)cc1] | 390.3 |
| 110 | 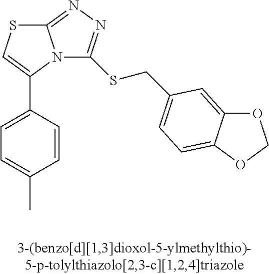 [SMILES: Cc1ccc(-c2csc3nnc(SCc4ccc5c(c4)OCO5)n23)cc1] | 382.4 |
| 111 | 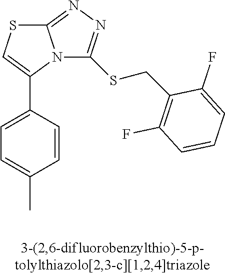 [SMILES: Cc1ccc(-c2csc3nnc(SCc4c(F)cccc4F)n23)cc1] | 374.4 |
| 112 | 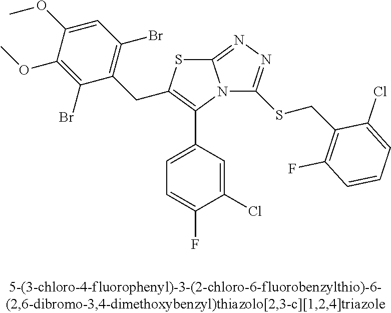 [SMILES: COc1cc(Br)c(Cc2sc3nnc(SCc4c(F)cccc4Cl)n3c2-c2ccc(F)c(Cl)c2)c(Br)c1OC] | 734.2 |
| 113 | 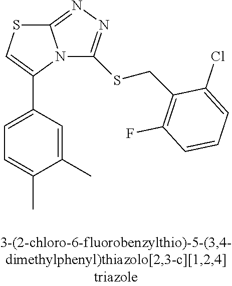 [SMILES: Cc1ccc(-c2csc3nnc(SCc4c(F)cccc4Cl)n23)cc1C] | 404.3 |
| 114 | 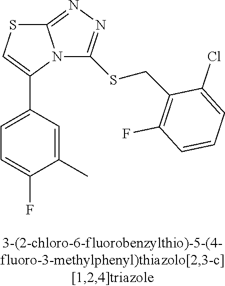 [SMILES: Cc1cc(-c2csc3nnc(SCc4c(F)cccc4Cl)n23)ccc1F] | 408.3 |
| 115 | 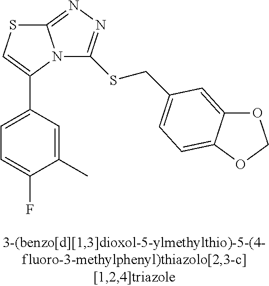 [SMILES: Cc1cc(-c2csc3nnc(SCc4ccc5c(c4)OCO5)n23)ccc1F] | 400.3 |
| 116 | 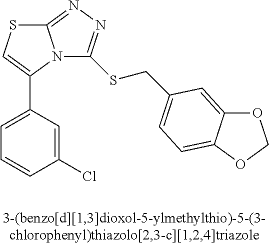 [SMILES: Clc1cccc(-c2csc3nnc(SCc4ccc5c(c4)OCO5)n23)c1] | 402.2 |
| 117 | 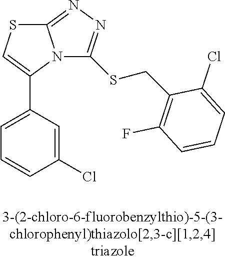 [SMILES: Fc1cccc(Cl)c1CSc1nnc2scc(-c3cccc(Cl)c3)n12] | 410.3 |
| 118 | 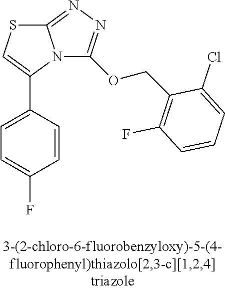 [SMILES: Fc1ccc(-c2csc3nnc(OCc4c(F)cccc4Cl)n23)cc1] | 378.3 |
| 119 | 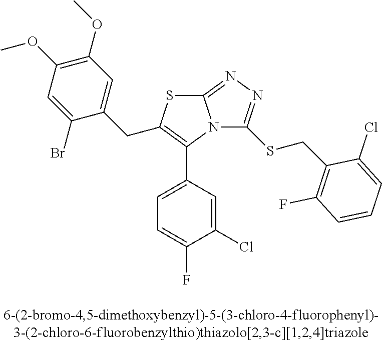 [SMILES: COc1cc(Br)c(Cc2sc3nnc(SCc4c(F)cccc4Cl)n3c2-c2ccc(F)c(Cl)c2)cc1OC] | 656.2 |
| 120 | 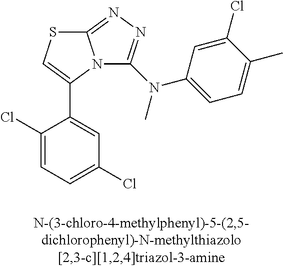 [SMILES: Cc1ccc(N(C)c2nnc3scc(-c4cc(Cl)ccc4Cl)n23)cc1Cl] | 423.3 |
| 121 | 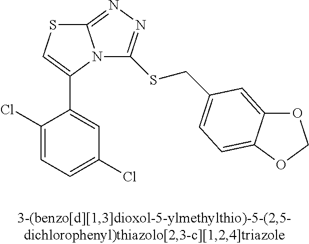 [SMILES: Clc1ccc(Cl)c(-c2csc3nnc(SCc4ccc5c(c4)OCO5)n23)c1] | 436.3 |
| 122 | 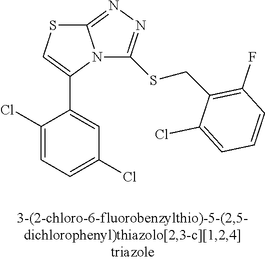 [SMILES: Fc1cccc(Cl)c1CSc1nnc2scc(-c3cc(Cl)ccc3Cl)n12] | 444.2 |
| 123 | 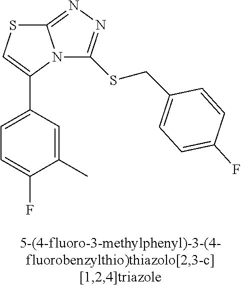 [SMILES: Cc1cc(-c2csc3nnc(SCc4ccc(F)cc4)n23)ccc1F] | 374.4 |
| 124 | 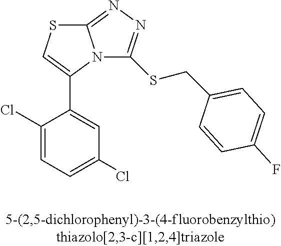 [SMILES: Fc1ccc(CSc2nnc3scc(-c4cc(Cl)ccc4Cl)n23)cc1] | 410.3 |
| 125 | 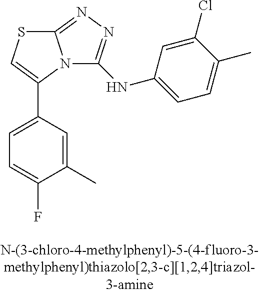 [SMILES: Cc1cc(-c2csc3nnc(Nc4ccc(C)c(Cl)c4)n23)ccc1F] | 373.4 |
| 126 | 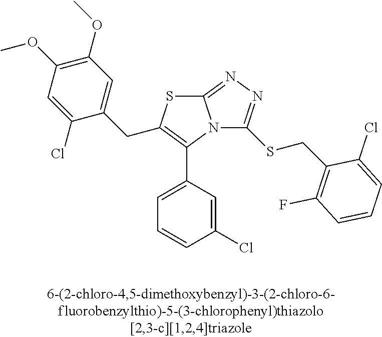 [SMILES: COc1cc(Cl)c(Cc2sc3nnc(SCc4c(F)cccc4Cl)n3c2-c2cccc(Cl)c2)cc1OC] | 594.3 |
| 127 | 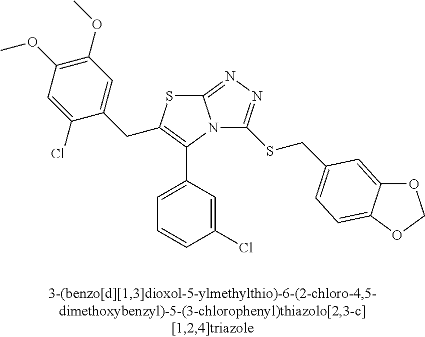 [SMILES: COc1cc(Cl)c(Cc2sc3nnc(SCc4ccc5c(c4)OCO5)n3c2-c2cccc(Cl)c2)cc1OC] | 586.3 |
| 128 | 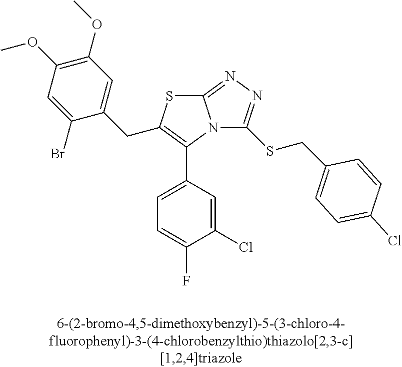 [SMILES: COc1cc(Br)c(Cc2sc3nnc(SCc4ccc(Cl)cc4)n3c2-c2ccc(F)c(Cl)c2)cc1OC] | 638.3 |
| 129 | 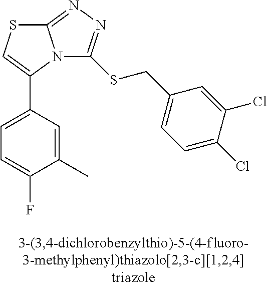 [SMILES: Cc1cc(-c2csc3nnc(SCc4ccc(Cl)c(Cl)c4)n23)ccc1F] | 424.3 |
| 130 | 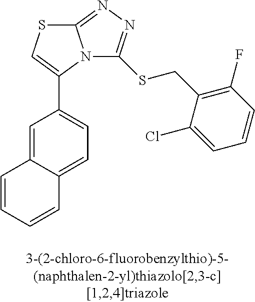 [SMILES: Fc1cccc(Cl)c1CSc1nnc2scc(-c3ccc4ccccc4c3)n12] | 426.3 |
| 131 | 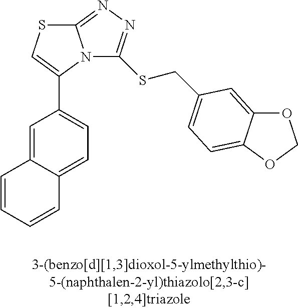 [SMILES: c1ccc2cc(-c3csc4nnc(SCc5ccc6c(c5)OCO6)n34)ccc2c1] | 418.3 |
| 132 | 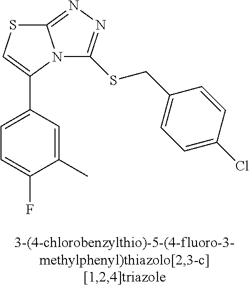 [SMILES: Cc1cc(-c2csc3nnc(SCc4ccc(Cl)cc4)n23)ccc1F] | 390.3 |
| 133 | 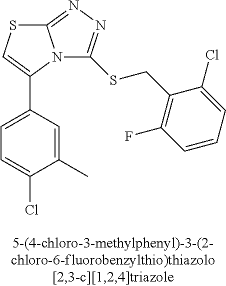 [SMILES: Cc1cc(-c2csc3nnc(SCc4c(F)cccc4Cl)n23)ccc1Cl] | 424.3 |
| 134 | 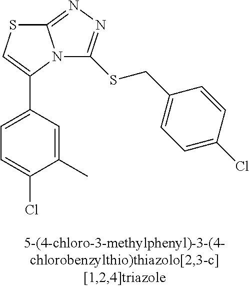 [SMILES: Cc1cc(-c2csc3nnc(SCc4ccc(Cl)cc4)n23)ccc1Cl] | 406.3 |
| 135 | 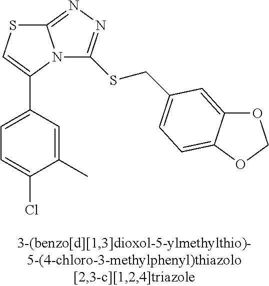 [SMILES: Cc1cc(-c2csc3nnc(SCc4ccc5c(c4)OCO5)n23)ccc1Cl] | 416.3 |
| 136 | 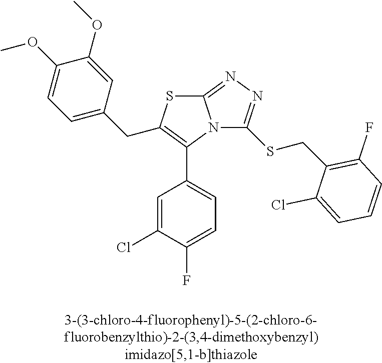 [SMILES: COc1ccc(Cc2sc3nnc(SCc4c(F)cccc4Cl)n3c2-c2ccc(F)c(Cl)c2)cc1OC] | 577.3 |
| 137 | 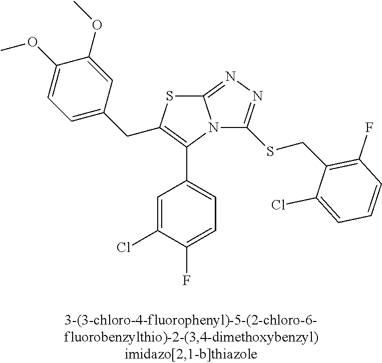 [SMILES: COc1ccc(Cc2sc3nnc(SCc4c(F)cccc4Cl)n3c2-c2ccc(F)c(Cl)c2)cc1OC] | 577.3 |

### Example 138

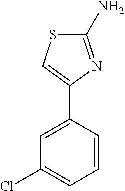 [SMILES: Nc1nc(-c2cccc(Cl)c2)cs1]

### 4-(3-chlorophenyl)thiazol-2-amine

Thiourea (0.81 g, 10.7 mmol) is added to 2-bromo-1-(3-chlorophenyl)ethanone (2.5 g, 10.7 mmol) in ethanol (25 mL) and the solution is refluxed for 6 hours. The mixture is then cooled and concentrated to half of the original volume at which time the product precipitates. The filtrate is then triturated with Et₂O and a second crop is collected. The two crops of the hydrobromide salt of 4-(3-chlorophenyl)thiazol-2-amine are then combined (2.0 g, 89%). LC/MS (m/z) M+H=211.2; t_{R}=2.2 min.

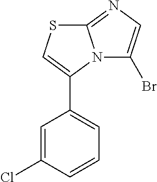 [SMILES: Clc1cccc(-c2csc3ncc(Br)n23)c1]

### 5-bromo-3-(3-chlorophenyl)imidazo[2,1-b]thiazole

2-Bromoacetaldehyde dimethylacetal (0.40 mL, 3.4 mmol) is added to a solution of 4-(3-chlorophenyl)thiazol-2-amine HBr salt (0.5 g, 1.7 mmol) in THF-water (1:1, 8 mL). The reaction is then stirred for 1 hour at room temperature followed by 6 hours at 90° C. The reaction is then cooled, diluted with EtOAc (100 mL) and washed with sodium bicarbonate solution (2×), brine. The organic phase is then dried over sodium sulfate and concentrated. LC/MS (m/z) M+H=235.3; t_{R}=2.1 min. NBS (0.30 g, 1.7 mmol) is then added to a mixture of the crude imidazole (1.7 mmol) in dichloromethane (17 mL) and the mixture is stirred overnight at room temperature. The mixture is concentrated and purified by chromatography to give the title compound (52 mg, 9%). LC/MS (m/z) M+H=315.3; t_{R}=2.9 min.

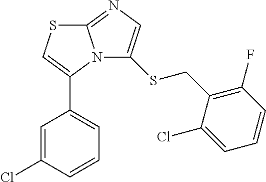 [SMILES: Fc1cccc(Cl)c1CSc1cnc2scc(-c3cccc(Cl)c3)n12]

### 5-[(2-chloro-6-fluoro-phenyl)methylsulfanyl]-3-(3-chlorophenyl)imidazo[2,1-b]thiazole

A mixture of (2-chloro-6-fluoro-phenyl)methanethiol (57 mg, 0.32 mmol), diisopropylethyl amine (0.056 mL, 0.32 mmol), Xantphos (19 mg, 0.03 mmol) and 5-bromo-3-(3-chlorophenyl)imidazo[2,1-b]thiazole (52 mg, 0.16 mmol) in 1,4-dioxane is charged in a microwave tube. To this is added Pd₂ dba₃ (8.0 mg, 0.008 mmol), and N₂ is bubbled through the solution for 1 minute and the vessel is sealed. Irradiation is followed for 40 minutes at 120° C. The solvent is removed and residue purified by preparative HPLC to afford the title compound (30 mg, 45.8 mg). LC/MS (m/z) M+H=409.3; t_{R}=3.5 min.

### Example 139

### Assay for the Identification of TGR5 Receptor Agonists Establishment of TGR5 Expressing Cell Line

Transformed E. coli stocks containing cDNA for the full length human TGR5 bile acid receptor (IMAGE clone #5221127) are obtained from Open Biosystems. Cultures are grown in LB broth, and cDNA is purified using Qiagen mini-prep columns. The full length TGR5 gene is inserted into pCMV6 vector DNA (Origene) by ligation followed by transformation in competent E. coli. Ampicillin-resistant clones are isolated and vector DNA containing the TGR5 gene is verified by DNA sequence analysis (Sequetech). The resulting plasmid is transfected into HEK 293 cells using FuGene 6 transfection reagent and the transfected cells are grown in the presence of 1 mg/mL G418 selection antibiotic. A number of individual G418 resistant colonies are isolated by using 150 μL cloning cylinders. The resulting 293/humanTGR5 expressing cell lines are then screened for responsiveness to the natural ligand lithocholic acid. A single clone is chosen for expansion and use for screening, this clone reproducibly responds to known receptor agonists for over 30 passages.

### Measurement of TGR5 Receptor Activation

The 293/humanTGR5 cells are maintained in DMEM/F12 media supplemented with 10% fetal bovine serum (Hyclone) and 15 mM HEPES buffer (Invitrogen). Cells are passaged twice weekly and maintained at densities to assure logarithmic growth. For use in cAMP assays, cells are trypsinzied, centrifuged, and resuspended in DMEM/F12 media supplemented with 0.5% FBA and 15 mM HEPES buffer. These cells are adjusted to a density which allows distribution of 25,000 cells in 20 μL of volume to each well of a half-area 96-well plate (Greiner Bio-One). 20 μL of test compounds diluted to a 2× concentration in DMEM/F12 media with 0.5% FBS are then added to the 96-well plates, and the plates are then incubated for 30 minutes at 37° C. in a 5% CO₂ atmosphere. Levels of resulting cAMP for each treatment condition are then determined by use of an HTRF cAMP assay (CisBio) following the manufactures directions. The resulting data is analyzed using IDBX XLfit software and IC₅₀ determinations are made for each test compound by comparing cAMP levels to vehicle controls (unstimulated) and TGR5 agonist-activated cells (50 μM of Compound 7, a reported TGR5 receptor agonist, J. Med. Chem., 2009, 52 (24), pp 7962-7965). Specificity for human TGR5 agonist activity is confirmed for compounds of interest by testing them against cell lines over expressing the mouse TGR5 receptor, human GPR119 receptor, and parent cell lines followed by measurement of resulting cAMP levels. Assay responsiveness is controlled for by measuring cAMP levels induced by 50 μM Forskolin in cells, a known adenylyl cylase activator.

Compounds of this invention are TGR5 bile acid receptor agonists. Selected exemplary compounds of the disclosure are listed in the table below with their activation activities on the 293/humanTGR5 cells measured by cAMP levels, where A represents an IC₅₀ value that is less than 0.5 μM, B represents an IC₅₀ value between 0.5 and 5 μM, and C represents an IC₅₀ value between 5 and 50 μM.

| Example | 293/humanTGR5 |
|---|---|
| No | cAMP |
| 19 | C |
| 24 | C |
| 30 | C |
| 46 | C |
| 48 | C |
| 2 | B |
| 8 | C |
| 59 | C |
| 65 | B |
| 69 | A |
| 76 | B |
| 83 | B |
| 17 | B |
| 97 | C |
| 11 | B |
| 100 | B |
| 109 | A |
| 115 | A |
| 119 | B |
| 120 | B |
| 127 | B |
| 18 | A |
| 6 | C |
| 26 | B |
| 43 | C |
| 7 | C |
| 4 | C |
| 50 | B |
| 53 | A |
| 1 | B |
| 66 | B |
| 3 | B |
| 80 | B |
| 86 | B |
| 94 | B |
| 98 | C |
| 12 | C |
| 106 | B |
| 112 | B |
| 117 | B |
| 14 | B |
| 15 | A |
| 131 | C |
| 138 | B |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A compound of the formula: 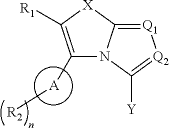
or a pharmaceutically acceptable salt thereof, wherein
X is O, S, or N(R₆);
Y is −OR₃, —O—Z—R₃, —SR₃, —S—Z—R₃, —N(R₆)R₃, —N(R₆)—Z—R₃, or —Z—R₃;
Z is —C(R₄)(R₅)—, or —C(R₄)(R₅)—C(R₄)(R₅)—;
Q₁ and Q₂ are independently N or CH, provided that at least one of Q₁ and Q₂ is N;
A is phenyl, naphthyl, pyridinyl, pyrazinyl, pyrimidinyl, or pyridazinyl;
n is 0, 1, 2, 3, or 4;
R₁ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, aryl, aryl C₁-C₆ alkyl, aryl C₁-C₆ alkenyl, heteroaryl, heteroaryl C₁-C₆ alkyl, heteroaryl C₁-C₆ alkenyl, —CON(C₁-C₆ alkyl)-aryl, —CONH-aryl, —O-aryl, —O—CH₂-aryl, —O—(CH₂)₂-aryl, —NH-aryl, —NH—CH₂-aryl, —NH—(CH₂)₂-aryl, —S-aryl, —S—CH₂-aryl, or —S—(CH₂)₂-aryl, wherein each is optionally substituted at any suitable position with one or more of R₇; or
each R₂ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —CONH₂, —CON(C₁-C₆ alkyl), —NHCO(C₁-C₆ alkyl), —N(C₁-C₆ alkyl)CO(C₁-C₆ alkyl), —CON(C₁-C₆ alkyl)₂, —CONH—OH, —CONH—NH₂, —CO₂H, —CO₂(C₁-C₆ alkyl), —OCO(C₁-C₆ alkyl), C₃-C₈ cycloalkyl, aryl, heteroaryl, and heterocyclyl;
R₃ is hydrogen, C₃-C₈ cycloalkyl, aryl, heteroaryl, or heterocyclyl, wherein each is optionally substituted with one or more of R₈;
each R₄ and R₅ independently are hydrogen, C₁-C₆ alkyl, or C₁-C₆ haloalkyl;
R₆ is hydrogen or C₁-C₆ alkyl;
R₇ is halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, or C₁-C₆ haloalkoxy; and
R₈ is halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —CONH₂, —CON(C₁-C₆ alkyl), —NHCO(C₁-C₆ alkyl), —N(C₁-C₆ alkyl)CO(C₁-C₆ alkyl), —CON(C₁-C₆ alkyl)₂, —CONH—OH, —CONH—NH₂, —CO₂H, —CO₂(C₁-C₆ alkyl), —OCO(C₁-C₆ alkyl), C₃-C₈ cycloalkyl, aryl, heteroaryl, or heterocyclyl;
provided the compound is not:
5-phenyl-3-(phenylmethyl)-thiazolo[2,3-c]-1,2,4-triazole;
5-(4-chlorophenyl)-3-(phenylmethyl)-thiazolo[2,3-c]-1,2,4-triazole;
5-(4-bromophenyl)-3-(phenylmethyl)-thiazolo[2,3-c]-1,2,4-triazole;
3-anilino-5,6-diphenyl-thiazolo[2,3-c]-s-triazole;
3-(benzylthio)-5,6-diphenyl-thiazolo[2,3-c]-s-triazole;
5-(4-fluorophenyl)-3-[(3-pyridinylmethyl)thio]-thiazolo[2,3-c]-1,2,4-triazole;
3-[(1,3-benzodioxol-5-ylmethyl)thio]-5-phenyl-thiazolo[2,3-c]-1,2,4-triazole;
5-(4-methylphenyl)-3-[[(2-methyl-4-thiazolyl)methyl]thio]-thiazolo[2,3-c]-1,2,4-triazole;
5-(4-chlorophenyl)-3-[[(3,5-dimethyl-4-isoxazolyl)methyl]thio]-thiazolo[2,3-c]-1,2,4-triazole;
3-[[(3,5-dimethyl-4-isoxazolyl)methyl]thio]-5-phenyl-thiazolo[2,3-c]-1,2,4-triazole;
5-(4-chlorophenyl)-3-[[(2-methyl-4-thiazolyl)methyl]thio]-thiazolo[2,3-c]-1,2,4-triazole;
3-[[(2-methyl-4-thiazolyl)methyl]thio]-5-phenyl-thiazolo[2,3-c]-1,2,4-triazole; or
3-anilino-5,6-diphenyl-oxazolo[2,3-c]-s-triazole.

2. A compound according to claim 1, of the formula:

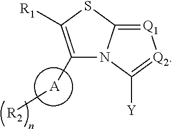

3. A compound according to claim 2, of the formula:

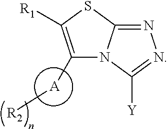

4. A compound according to claim 3, of the formula:

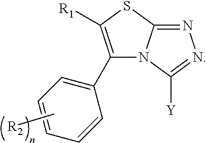

5. A compound according to claim 3, of the formula:

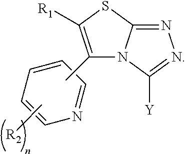

6. A compound according to claim 1, of the formula:

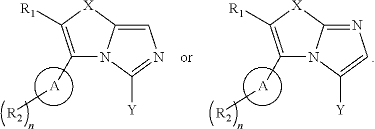

7. A compound according to claim 6, of the formula:

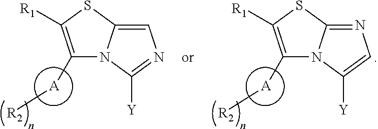

8. A compound according to claim 7, of the formula:

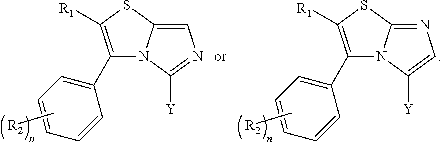

9. A compound according to claim 7, of the formula:

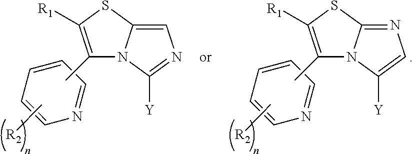

10. A compound according to claim 1, wherein
R₁ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, aryl, aryl C₁-C₆ alkyl, aryl C₁-C₆ alkenyl, heteroaryl, heteroaryl C₁-C₆ alkyl, —CON(C₁-C₆ alkyl)-aryl, —CONH-aryl, —O—CH₂-aryl, —NH—CH₂-aryl, or —S—CH₂-aryl, wherein each is optionally substituted at any suitable position with one or more of R₇.

11. A compound according to claim 10, wherein R₁ is hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, aryl, aryl C₁-C₆ alkyl, aryl C₁-C₆ alkenyl, heteroaryl, or heteroaryl C₁-C₆ alkyl, wherein each is optionally substituted at any suitable position with one or more of R₇.

12. A compound according to claim 11, wherein R₁ is hydrogen, halogen, C₁-C₆ alkyl, aryl, aryl C₁-C₆ alkyl, aryl C₁-C₆ alkenyl, heteroaryl, or heteroaryl C₁-C₆ alkyl, wherein each is optionally substituted at any suitable position with one or more of R₇.

13. A compound according to claim 12, wherein R₁ is hydrogen, halogen, or C₁-C₆ alkyl.

14. A compound according to claim 13, wherein R₁ is hydrogen.

15. A compound according to claim 12, wherein R₁ is aryl, aryl C₁-C₆ alkyl, or aryl C₁-C₆ alkenyl, wherein each is optionally substituted at any suitable position with one or more of R₇.

16. A compound according to claim 1, which is:
3-(2-chloro-6-fluorobenzylthio)-5-phenylthiazolo[2,3-c][1,2,4]triazole;
3-(benzo[d][1,3]dioxol-5-ylmethylthio)-5-phenylthiazolo[2,3-c][1,2,4]triazole;
3-(3,5-dimethoxybenzylthio)-5-phenylthiazolo[2,3-c][1,2,4]triazole;
3-(4-fluorobenzylthio)-5-phenylthiazolo[2,3-c][1,2,4]triazole;
5-phenyl-3-(4-(trifluoromethyl)benzylthio)thiazolo[2,3-c][1,2,4]triazole;
3-(benzo[d][1,3]dioxol-5-ylmethylthio)-5-(4-chlorophenyl)thiazolo[2,3-c][1,2,4]triazole;
3-(benzo[d][1,3]dioxol-5-ylmethylthio)-5-(4-fluorophenyl)thiazolo[2,3-c][1,2,4]triazole;
3-((5-phenylthiazolo[2,3-c][1,2,4]triazol-3-ylthio)methyl)phenol;
3-(4-chlorobenzylthio)-5-phenylthiazolo[2,3-c][1,2,4]triazole;
3-(3-chloro-4-methoxybenzylthio)-5-phenylthiazolo[2,3-c][1,2,4]triazole;
3-(2,4-difluorobenzylthio)-5-phenylthiazolo[2,3-c][1,2,4]triazole;
5-phenyl-3-(2-(trifluoromethyl)benzylthio)thiazolo[2,3-c][1,2,4]triazole;
3-(phenethylthio)-5-phenylthiazolo[2,3-c][1,2,4]triazole;
3-(4-chlorophenethylthio)-5-phenylthiazolo[2,3-c][1,2,4]triazole;
3-(3,4-dimethoxyphenethylthio)-5-phenylthiazolo[2,3-c][1,2,4]triazole;
5-phenyl-3-(4-(trifluoromethoxy)benzylthio)thiazolo[2,3-c][1,2,4]triazole;
5-phenyl-3-(3-(trifluoromethyl)benzylthio)thiazolo[2,3-c][1,2,4]triazole;
3-(benzo[d][1,3]dioxol-5-ylmethylthio)-5-(2-chlorophenyl)thiazolo[2,3-c][1,2,4]triazole;
3-(4-methoxybenzylthio)-5-phenylthiazolo[2,3-c][1,2,4]triazole;
3-((2,3-dihydrobenzo[b][1,4]dioxin-6-yl)methylthio)-5-phenylthiazolo[2,3-c][1,2,4]triazole;
3-(3-fluoro-4-methoxybenzylthio)-5-phenylthiazolo[2,3-c][1,2,4]triazole;
3-((2,3-dihydro-1H-inden-5-yl)methylthio)-5-phenylthiazolo[2,3-c][1,2,4]triazole;
3-(isoquinolin-5-ylmethylthio)-5-phenylthiazolo[2,3-c][1,2,4]triazole;
3-((6-chlorobenzo[d][1,3]dioxol-5-yl)methylthio)-5-phenylthiazolo[2,3-c][1,2,4]triazole;
3-(benzo[d][1,3]dioxol-5-ylmethylthio)-6-methyl-5-phenylthiazolo[2,3-c][1,2,4]triazole;
3-(2-chlorobenzylthio)-5-(2-chlorophenyl)thiazolo[2,3-c][1,2,4]triazole;
3-(isoquinolin-6-ylmethylthio)-5-phenylthiazolo[2,3-c][1,2,4]triazole;
3-(benzo[d][1,3]dioxol-5-ylthio)-5-phenylthiazolo[2,3-c][1,2,4]triazole;
3-(2-chloro-6-fluorophenylthio)-5-phenylthiazolo[2,3-c][1,2,4]triazole;
3-(benzo[d][1,3]dioxol-5-yloxy)-5-phenylthiazolo[2,3-c][1,2,4]triazole;
3-((1H-benzo[d]imidazol-5-yl)methoxy)-5-phenylthiazolo[2,3-c][1,2,4]triazole;
(3-(benzo[d][1,3]dioxol-5-ylmethylthio)-5-phenylthiazolo[2,3-c][1,2,4]triazol-6-yl)(4-fluorophenyl)methanol;
3-(benzo[d][1,3]dioxol-5-ylmethoxy)-5-phenylthiazolo[2,3-c][1,2,4]triazole;
3-(benzo[d][1,3]dioxol-5-ylmethylthio)-6-ethyl-5-phenylthiazolo[2,3-c][1,2,4]triazole;
3-(2-(benzo[d][1,3]dioxol-5-yl)ethyl)-5-phenylthiazolo[2,3-c][1,2,4]triazole;
tert-butyl 3-chloro-5-fluoro-4-((5-(4-fluorophenyl)thiazolo[2,3-c][1,2,4]triazol-3-ylthio)methyl)benzoate;
N-(3-chloro-4-methylphenyl)-N,3-dimethyl-5-phenylthiazolo[2,3-c][1,2,4]triazole-6-carboxamide;
3-(benzo[d][1,3]dioxol-5-ylmethylthio)-5-(2,4-difluorophenyl)thiazolo[2,3-c][1,2,4]triazole;
3-(benzo[d][1,3]dioxol-5-ylmethylthio)-5-(4-fluoro-3-methoxyphenyl)thiazolo[2,3-c][1,2,4]triazole;
6-(2-chloro-6-fluorobenzylthio)-3-methyl-5-phenylthiazolo[2,3-c][1,2,4]triazole;
3-(benzo[d][1,3]dioxol-5-ylmethylthio)-5-(4-fluoro-2-methoxyphenyl)thiazolo[2,3-c][1,2,4]triazole;
3-(benzo[d][1,3]dioxol-5-ylmethylthio)-5-(2-bromo-4-fluorophenyl)thiazolo[2,3-c][1,2,4]triazole;
3-(benzo[d][1,3]dioxol-5-ylmethylthio)-5-(3-bromo-4-fluorophenyl)thiazolo[2,3-c][1,2,4]triazole;
3-(benzo[d][1,3]dioxol-5-ylmethylthio)-5-(4-fluoro-3-(trifluoromethyl)phenyl)thiazolo[2,3-c][1,2,4]triazole;
3-(benzo[d][1,3]dioxol-5-ylmethylthio)-5-(3-chloro-4-fluorophenyl)thiazolo[2,3-c][1,2,4]triazole;
3-(benzo[d][1,3]dioxol-5-ylmethylthio)-5-(4-fluoro-2-nitrophenyl)thiazolo[2,3-c][1,2,4]triazole;
2-(3-(benzo[d][1,3]dioxol-5-ylmethylthio)thiazolo[2,3-c][1,2,4]triazol-5-yl)-5-fluoroaniline;
2-(3-(benzo[d][1,3]dioxol-5-ylmethylthio)thiazolo[2,3-c][1,2,4]triazol-5-yl)-5-fluoro-N,N-dimethylaniline;
N-(2-(3-(benzo[d][1,3]dioxol-5-ylmethylthio)thiazolo[2,3-c][1,2,4]triazol-5-yl)-5-fluorophenyl)-N-methylpivalamide;
3-(benzo[d][1,3]dioxol-5-ylmethylthio)-5-(2-chloro-4-fluorophenyl)thiazolo[2,3-c][1,2,4]triazole;
N-(3-chloro-4-methylphenyl)-N-methyl-5-phenylthiazolo[2,3-c][1,2,4]triazole-6-carboxamide;
3-(benzo[d][1,3]dioxol-5-ylmethylthio)-5-(3-nitrophenyl)thiazolo[2,3-c][1,2,4]triazole;
3-(3-(benzo[d][1,3]dioxol-5-ylmethylthio)thiazolo[2,3-c][1,2,4]triazol-5-yl)benzonitrile;
3-(benzo[d][1,3]dioxol-5-ylmethylthio)-5-(3-isopropylphenyl)thiazolo[2,3-c][1,2,4]triazole;
3-(benzo[d][1,3]dioxol-5-ylmethylthio)-5-(5-chloro-2-methoxyphenyl)thiazolo[2,3-c][1,2,4]triazole;
3-(benzo[d][1,3]dioxol-5-ylmethylthio)-5-(3-tert-butylphenyl)thiazolo[2,3-c][1,2,4]triazole;
3-(benzo[d][1,3]dioxol-5-ylmethylthio)-5-(2-chloro-4-methoxyphenyl)thiazolo[2,3-c][1,2,4]triazole;
methyl 3-((5-(3-bromophenyl)thiazolo[2,3-c][1,2,4]triazol-3-ylthio)methyl)benzoate;
5-(3-bromophenyl)-3-(3,4-dichlorobenzylthio)thiazolo[2,3-c][1,2,4]triazole;
5-(3-bromophenyl)-3-(2-chloro-4-fluorobenzylthio)thiazolo[2,3-c][1,2,4]triazole;
3-(benzo[d][1,3]dioxol-5-ylmethylthio)-5-(3-bromophenyl)thiazolo[2,3-c][1,2,4]triazole;
3-(benzo[d][1,3]dioxol-5-ylmethylthio)-N-(3-chloro-4-methylphenyl)-N-methyl-5-phenylthiazolo[2,3-c][1,2,4]triazole-6-carboxamide;
3-(benzo[d][1,3]dioxol-5-ylmethylthio)-5-(3-chloro-2-methylphenyl)thiazolo[2,3-c][1,2,4]triazole;
3-(benzo[d][1,3]dioxol-5-ylmethylthio)-5-(5-chloro-2-methylphenyl)thiazolo[2,3-c][1,2,4]triazole;
3-(benzo[d][1,3]dioxol-5-ylmethylthio)-5-(3,4-difluorophenyl)thiazolo[2,3-c][1,2,4]triazole;
3-(benzo[d][1,3]dioxol-5-ylmethylthio)-6-(2-bromo-4,5-dimethoxybenzyl)-5-(4-fluorophenyl)thiazolo[2,3-c][1,2,4]triazole;
6-(2-bromo-4,5-dimethoxybenzyl)-3-(2-chloro-6-fluorobenzylthio)-5-(4-fluorophenyl)thiazolo[2,3-c][1,2,4]triazole;
3-(benzo[d][1,3]dioxol-5-ylmethylthio)-5-(2,4-dichlorophenyl)thiazolo[2,3-c][1,2,4]triazole;
3-(benzo[d][1,3]dioxol-5-ylmethylthio)-5-(2,3-dichlorophenyl)thiazolo[2,3-c][1,2,4]triazole;
3-(benzo[d][1,3]dioxol-5-ylmethylthio)-5-(3-chloro-5-(trifluoromethyl)phenyl)thiazolo[2,3-c][1,2,4]triazole;
3-(benzo[d][1,3]dioxol-5-ylmethylthio)-5-(4-nitrophenyl)thiazolo[2,3-c][1,2,4]triazole;
3-(benzo[d][1,3]dioxol-5-ylmethylthio)-5-(biphenyl-3-yl)thiazolo[2,3-c][1,2,4]triazole;
3-(benzo[d][1,3]dioxol-5-ylmethylthio)-5-(3-cyclopropylphenyl)thiazolo[2,3-c][1,2,4]triazole;
3-(benzo[d][1,3]dioxol-5-ylmethylthio)-5-m-tolylthiazolo[2,3-c][1,2,4]triazole;
3-(benzo[d][1,3]dioxol-5-ylmethylthio)-6-(3,4-dimethoxybenzyl)-5-(4-fluorophenyl)thiazolo[2,3-c][1,2,4]triazole;
3-(2-chloro-6-fluorobenzylthio)-6-(3,4-dimethoxybenzyl)-5-(4-fluorophenyl)thiazolo[2,3-c][1,2,4]triazole;
3-(2-chloro-6-fluorobenzylthio)-6-(4-chlorobenzyl)-5-(4-fluorophenyl)thiazolo[2,3-c][1,2,4]triazole;
3-(benzo[d][1,3]dioxol-5-ylmethylthio)-5-(3-chloro-4-methylphenyl)thiazolo[2,3-c][1,2,4]triazole;
5-(3-chloro-4-methylphenyl)-3-(2-chloro-6-fluorobenzylthio)thiazolo[2,3-c][1,2,4]triazole;
3-(2-chloro-6-fluorobenzylthio)-5-(4-fluorophenyl)thiazolo[2,3-c][1,2,4]triazole;
6-bromo-3-(2-chloro-6-fluorobenzylthio)-5-(4-fluorophenyl)thiazolo[2,3-c][1,2,4]triazole;
3-(2-chloro-6-fluorobenzylthio)-5-(3-chlorophenyl)-6-(3,4-dimethoxybenzyl)thiazolo[2,3-c][1,2,4]triazole;
3-(benzo[d][1,3]dioxol-5-ylmethylthio)-5-(3-chlorophenyl)-6-(3,4-dimethoxybenzyl)thiazolo[2,3-c][1,2,4]triazole;
tert-butyl 3-chloro-4-((5-(3-chlorophenyl)-6-(3,4-dimethoxybenzyl)thiazolo[2,3-c][1,2,4]triazol-3-ylthio)methyl)-5-fluorobenzoate;
3-(2-chloro-6-fluorobenzylthio)-6-(2-chloro-6-fluorophenyl)-5-(4-fluorophenyl)thiazolo[2,3-c][1,2,4]triazole;
3-(benzo[d][1,3]dioxol-5-ylmethylthio)-6-(2-chloro-6-fluorophenyl)-5-(4-fluorophenyl)thiazolo[2,3-c][1,2,4]triazole;
6-(2-chloro-6-fluorophenyl)-3-(3,4-dichlorobenzylthio)-5-(4-fluorophenyl)thiazolo[2,3-c][1,2,4]triazole;
6-(2-chloro-6-fluorophenyl)-3-(3-fluoro-4-methoxybenzylthio)-5-(4-fluorophenyl)thiazolo[2,3-c][1,2,4]triazole;
3-(3-chloro-4-fluorobenzylthio)-6-(2-chloro-6-fluorophenyl)-5-(4-fluorophenyl)thiazolo[2,3-c][1,2,4]triazole;
6-(2-chloro-6-fluorophenyl)-3-(3,4-dimethoxybenzylthio)-5-(4-fluorophenyl)thiazolo[2,3-c][1,2,4]triazole;
3-(3-chloro-4-methoxybenzylthio)-6-(2-chloro-6-fluorophenyl)-5-(4-fluorophenyl)thiazolo[2,3-c][1,2,4]triazole;
3-(benzo[d][1,3]dioxol-5-ylmethyl)-6-(2-chloro-6-fluorophenyl)-5-(4-fluorophenyl)thiazolo[2,3-c][1,2,4]triazole;
3-(2-chloro-6-fluorobenzylthio)-5-(4-fluorophenyl)-6-(1-phenylethyl)thiazolo[2,3-c][1,2,4]triazole;
3-(2-chloro-6-fluorobenzylthio)-6-(2-(3,4-dimethoxyphenyl)propan-2-yl)-5-phenylthiazolo[2,3-c][1,2,4]triazole;
3-(2-chloro-6-fluorobenzylthio)-6-(1-(3,4-dimethoxyphenyl)vinyl)-5-phenylthiazolo[2,3-c][1,2,4]triazole;
6-(3-chloro-4-methoxybenzyl)-3-(2-chloro-6-fluorobenzylthio)-5-(4-fluorophenyl)thiazolo[2,3-c][1,2,4]triazole;
5-(3-chloro-4-fluorophenyl)-6-(3-chloro-4-methoxybenzyl)-3-(2-chloro-6-fluorobenzylthio)thiazolo[2,3-c][1,2,4]triazole;
3-(benzo[d][1,3]dioxol-5-ylmethylthio)-5-(3-chloro-4-fluorophenyl)-6-(3-chloro-4-methoxybenzyl)thiazolo[2,3-c][1,2,4]triazole;
6-(2-chloro-6-fluorobenzyl)-3-(2-chloro-6-fluorobenzylthio)-5-phenylthiazolo[2,3-c][1,2,4]triazole;
5-(3-chloro-4-fluorophenyl)-6-(3-chloro-4-methoxybenzyl)-3-(2,6-difluorobenzylthio)thiazolo[2,3-c][1,2,4]triazole;
5-(3-chloro-4-fluorophenyl)-6-(3-chloro-4-methoxybenzyl)-3-(2,6-dichlorobenzylthio)thiazolo[2,3-c][1,2,4]triazole;
3-(2-chloro-4-fluorobenzylthio)-5-(3-chloro-4-fluorophenyl)-6-(3-chloro-4-methoxybenzyl)thiazolo[2,3-c][1,2,4]triazole;
5-(3-chloro-4-fluorophenyl)-6-(3-chloro-4-methoxybenzyl)-3-(2,4-difluorobenzylthio)thiazolo[2,3-c][1,2,4]triazole;
3-(3-chloro-4-fluorobenzylthio)-5-(3-chloro-4-fluorophenyl)-6-(3-chloro-4-methoxybenzyl)thiazolo[2,3-c][1,2,4]triazole;
3-(biphenyl-2-ylmethylthio)-5-(3-chloro-4-fluorophenyl)-6-(3-chloro-4-methoxybenzyl)thiazolo[2,3-c][1,2,4]triazole;
3-(2-chloro-6-fluorobenzylthio)-5-p-tolylthiazolo[2,3-c][1,2,4]triazole;
3-(benzo[d][1,3]dioxol-5-ylmethylthio)-5-p-tolylthiazolo[2,3-c][1,2,4]triazole;
3-(2,6-difluorobenzylthio)-5-p-tolylthiazolo[2,3-c][1,2,4]triazole;
5-(3-chloro-4-fluorophenyl)-3-(2-chloro-6-fluorobenzylthio)-6-(2,6-dibromo-3,4-dimethoxybenzyl)thiazolo[2,3-c][1,2,4]triazole;
3-(2-chloro-6-fluorobenzylthio)-5-(3,4-dimethylphenyl)thiazolo[2,3-c][1,2,4]triazole;
3-(2-chloro-6-fluorobenzylthio)-5-(4-fluoro-3-methylphenyl)thiazolo[2,3-c][1,2,4]triazole;
3-(benzo[d][1,3]dioxol-5-ylmethylthio)-5-(4-fluoro-3-methylphenyl)thiazolo[2,3-c][1,2,4]triazole;
3-(benzo[d][1,3]dioxol-5-ylmethylthio)-5-(3-chlorophenyl)thiazolo[2,3-c][1,2,4]triazole;
3-(2-chloro-6-fluorobenzylthio)-5-(3-chlorophenyl)thiazolo[2,3-c][1,2,4]triazole;
3-(2-chloro-6-fluorobenzyloxy)-5-(4-fluorophenyl)thiazolo[2,3-c][1,2,4]triazole;
6-(2-bromo-4,5-dimethoxybenzyl)-5-(3-chloro-4-fluorophenyl)-3-(2-chloro-6-fluorobenzylthio)thiazolo[2,3-c][1,2,4]triazole;
N-(3-chloro-4-methylphenyl)-5-(2,5-dichlorophenyl)thiazolo[2,3-c][1,2,4]triazol-3-amine;
N-(3-chloro-4-methylphenyl)-5-(2,5-dichlorophenyl)-N-methylthiazolo[2,3-c][1,2,4]triazol-3-amine;
5-(2-chloro-6-fluorobenzylthio)-3-(3-chlorophenyl)imidazo[5,1-b]thiazole;
3-(benzo[d][1,3]dioxol-5-ylmethylthio)-5-(2,5-dichlorophenyl)thiazolo[2,3-c][1,2,4]triazole;
3-(2-chloro-6-fluorobenzylthio)-5-(2,5-dichlorophenyl)thiazolo[2,3-c][1,2,4]triazole;
5-(4-fluoro-3-methylphenyl)-3-(4-fluorobenzylthio)thiazolo[2,3-c][1,2,4]triazole;
5-(2,5-dichlorophenyl)-3-(4-fluorobenzylthio)thiazolo[2,3-c][1,2,4]triazole;
N-(3-chloro-4-methylphenyl)-5-(4-fluoro-3-methylphenyl)thiazolo[2,3-c][1,2,4]triazol-3-amine;
6-(2-chloro-4,5-dimethoxybenzyl)-3-(2-chloro-6-fluorobenzylthio)-5-(3-chlorophenyl)thiazolo[2,3-c][1,2,4]triazole;
3-(benzo[d][1,3]dioxol-5-ylmethylthio)-6-(2-chloro-4,5-dimethoxybenzyl)-5-(3-chlorophenyl)thiazolo[2,3-c][1,2,4]triazole;
6-(2-bromo-4,5-dimethoxybenzyl)-5-(3-chloro-4-fluorophenyl)-3-(4-chlorobenzylthio)thiazolo[2,3-c][1,2,4]triazole;
3-(3,4-dichlorobenzylthio)-5-(4-fluoro-3-methylphenyl)thiazolo[2,3-c][1,2,4]triazole;
3-(2-chloro-6-fluorobenzylthio)-5-(naphthalen-2-yl)thiazolo[2,3-c][1,2,4]triazole;
3-(benzo[d][1,3]dioxol-5-ylmethylthio)-5-(naphthalen-2-yl)thiazolo[2,3-c][1,2,4]triazole;
3-(4-chlorobenzylthio)-5-(4-fluoro-3-methylphenyl)thiazolo[2,3-c][1,2,4]triazole;
5-(2-chloro-6-fluorobenzylthio)-3-(3-chlorophenyl)-2-(3,4-dimethoxybenzyl)imidazo[5,1-b]thiazole;
5-(4-chloro-3-methylphenyl)-3-(2-chloro-6-fluorobenzylthio)thiazolo[2,3-c][1,2,4]triazole;
5-(4-chloro-3-methylphenyl)-3-(4-chlorobenzylthio)thiazolo[2,3-c][1,2,4]triazole;
3-(benzo[d][1,3]dioxol-5-ylmethylthio)-5-(4-chloro-3-methylphenyl)thiazolo[2,3-c][1,2,4]triazole;
3-(3-chloro-4-fluorophenyl)-5-(2-chloro-6-fluorobenzylthio)-2-(3,4-dimethoxybenzyl)imidazo[5,1-b]thiazole;
3-(3-chloro-4-fluorophenyl)-5-(2-chloro-6-fluorobenzylthio)-2-(3,4-dimethoxybenzyl)imidazo[2,1-b]thiazole;
5-(2-chloro-6-fluorobenzylthio)-3-(3-chlorophenyl)imidazo[2,1-b]thiazole; or
pharmaceutically acceptable salts thereof.

17. A pharmaceutical composition comprising one or more compounds according to claim 1 and a pharmaceutically acceptable carrier, solvent, adjuvant or diluent.

18. A method of treating obesity or type II diabetes, the method comprising administering to a subject in need of such treatment an effective amount of one or more compounds according to claim 1.

19. A method of lowering blood glucose, the method comprising administering to a subject in need of such treatment an effective amount of one or more compounds according to claim 1.

20. A method for enhancing insulin secretion, the method comprising administering to a subject in need of such treatment an effective amount of one or more compounds according to claim 1.
